(19) United States
(12) Patent Application Publication
(10) Pub. No.: US 2024/0081892 A1
(43) Pub. Date: Mar. 14, 2024

(54) ELECTROSURGICAL TISSUE SEALING DEVICE WITH NON-STICK COATING

(71) Applicant: GYRUS ACMI, INC. D/B/A OLYMPUS SURGICAL TECHNOLOGIES AMERICA, Westborough, MA (US)

(72) Inventors: Kester Julian Batchelor, Mound, MN (US); Teo Heng Jimmy Yang, Heath (GB); Riyad Moe, Madison, WI (US)

(21) Appl. No.: 18/262,313

(22) PCT Filed: Jan. 27, 2022
(86) PCT No.: PCT/US2022/014098

§ 371 Date: Jul. 20, 2023

Related U.S. Application Data
(60) Provisional application No. 63/143,353, filed on Jan. 29, 2021.
(60) Provisional application No. 63/175,910, filed on Apr. 16, 2021.
(60) Provisional application No. 63/270,966, filed on Oct. 22, 2021.

(51) Int. Cl. A61B 18/14

(52) U.S. Cl. CPC A61B 18/1445; A61B 2018/0013

(57) ABSTRACT

An electrosurgical instrument includes a jaw member having an electrically conductive tissue sealing plate configured to operably couple to a source of electrosurgical energy for treating tissue. A liquidphobic structure is added to at least a portion of the electrically conductive sealing plate to reduce tissue adherence during application of electrical energy to tissue.

### CROSS REFERENCE TO RELATED APPLICATION

This application claims priority to U.S. Provisional Patent Application No. 63/143,353, titled “ELECTROSURGICAL TISSUE SEALING DEVICE WITH NON-STICK COATING”, filed on Jan. 29, 2021, the contents of which are hereby incorporated by reference. This application also claims priority to U.S. Provisional Patent Application No. 63/175,910, titled “COATINGS FOR ELECTROSURGICAL ELEMENTS”, filed on Apr. 16, 2021, the contents of which are hereby incorporated by reference. This application also claims priority to U.S. Provisional Patent Application No. 63/270,966, titled “ELECTROSURGICAL TISSUE SEALING DEVICE WITH NON-STICK COATING”, filed on Oct. 22, 2021, the contents of which are hereby incorporated by reference.

### OVERVIEW

Electrosurgical forceps utilize mechanical clamping action along with electrical energy to effect hemostasis on the clamped tissue. The forceps (open, laparoscopic or endoscopic) include electrosurgical sealing plates which apply the electrosurgical energy to the clamped tissue. By controlling the intensity, frequency, and duration of the electrosurgical energy applied through the sealing plates to the tissue, the surgeon can coagulate, cauterize, and/or seal tissue.

Improvements in electrosurgical instruments and the like, with a view towards providing improved transmission of electrical energy to patient tissue in both an effective manner and to reduce the sticking of soft tissue to the instrument's surface during application are desired. In general, efforts have envisioned non-stick surface coatings, such as polymeric materials, for increasing the lubricity of the tool surface. However, these materials may interfere with the efficacy and efficiency of hemostasis and have a tendency to release from the instrument's substrate due to formation of microporosity, delamination, and/or abrasive wear, thus exposing underlying portions of the instrument to direct tissue contact and related sticking issues. In turn, these holes or voids in the coating lead to nonuniform variations in the capacitive transmission of the electrical energy to the tissue of the patient and may create localized excess heating, resulting in tissue damage, undesired irregular sticking of tissue to the electrodes.

The present inventors have recognized, among other things, that problems to be solved in using electrosurgical devices is to provide a non-stick coating to minimize undesired irregular sticking or damage to tissue while providing benefits. For example, the present inventors have recognized that providing a non-stick coating formed from a liquidphobic structure can provide tissue adhesion resistance.

In one example of the present disclosure, an electrosurgical instrument is provided and includes at least one jaw member having an electrically conductive tissue sealing plate configured to operably couple to a source of electrosurgical energy for treating tissue and a non-stick coating formed from a liquidphobic structure.

In one example, the liquidphobic structure can be formed into the substrate, e.g., a portion of the electrically conductive tissue sealing plate. For example, a portion of the electrosurgical device can be formed to include a textured portion. The textured portion can include one of nanostructures, microstructures, and a hierarchical structure including nanostructures superimposed on microstructures. As discussed herein, the textured portion can include a micro-scale topography, a nano-scale topography, or a nano-scale topography superimposed on the micro-scale topography. The micro-scale topography can include micropillars and the nano-scale topography can include nanopitting or tube-like structures nanosuperimposed on the micro-pillars to impart hydrophobicity or superhydrophobicity to reduce the tissue adherence when electrosurgical energy is applied to tissue.

The textured portion can be formed during manufacturing, e.g., during molding, or can be formed after manufacturing as a post-treatment. For example, the post-treatments can include laser etching, chemical etching, and micro machining, among others.

In one example, liquidphobic structure can be an additional material that is deposited onto the substrate such as a coating. Various coatings are contemplated. In one example, the coating can be selected from at least one of tungsten disulfide (WS2), siloxanes such as hexamethyldisiloxane (HMDSO), polydimethylsiloxane (PDMSO), and tetramethyldisiloxane (TMDSO), fluorosilane containing compounds, glass, perfluoropolyether derivates, nanotube coatings, and other hydrophobic and superhydrophobic coatings.

In one example, the liquidphobic structure includes both the textured portion of the substrate, with the textured portion of the substrate including the coating. For example, the textured portion of the electrically conductive tissue sealing plate can include the coatings as described herein. Additionally, in one example, the electrically conducive tissue sealing plate can include a first liquidphobic structure and other portions of the device such as the polymeric body can include a second liquidphobic structure that is different from the first liquidphobic structure.

In one example of the present disclosure, an electrosurgical instrument is provided and includes at least one jaw member having an electrically conductive tissue sealing plate configured to operably couple to a source of electrosurgical energy for treating tissue and a non-stick coating formed from a liquidphobic structure. In one example, the liquidphobic structure can be the textured surface including micro-pillars on the electrically conductive tissue sealing plate. The micro-pillars can have a diameter within a range of about 70 microns to about 250, a height within a range of about 10 microns to about 100 microns, and a center-to-center spacing of about 120 microns to about 300 microns. The textured surface allow for air gaps to form under a contact liquid thereby a superhydrophobic property is created.

In one example, the non-stick coating has a substantially uniform thickness. In another example, the non-stick coating has a non-uniform thickness. In another example, the non-stick coating is discontinuous. In another example, the non-stick coating is continuous. In another example, the electrosurgical instrument also includes an insulative layer disposed on at least a portion of the tissue sealing plate. In another example, the non-stick coating is disposed on at least a portion of each of the pair of opposing jaw members. In another example, the tissue sealing plate is formed of stainless steel. In an example including the textured substrate, the density of the textured surface can be uniform or nonuniform.

According to another example of the present disclosure, an electrosurgical instrument is provided and includes a pair of opposing jaw members. Each of the opposing jaw members includes an electrically conductive tissue sealing plate configured to operably couple to a source of electrosurgical energy for treating tissue, a support base configured to support the tissue sealing plate, and an insulative housing configured to secure the tissue sealing plate to the support base. A non-stick coating formed from a liquidphobic structure is disposed on at least a portion of at least one of the opposing jaw members. In one example, the non-stick coating is disposed on at least a portion of each of the tissue sealing plates, the support base, and the insulative housing. In one example, the non-stick coating thickness has a substantially uniform thickness on the tissues sealing plates, the support base, and the insulative housing. another example, the coating has a non-uniform thickness. In another example, the non-stick coating is discontinuous. In another example, the non-stick coating is continuous.

According to another example of the present disclosure, an electrosurgical instrument is provided and includes a pair of opposing jaw members. Each of the opposing jaw members includes an electrically conductive tissue sealing plate configured to operably couple to a source of electrosurgical energy for treating tissue, a support base configured to support the tissue sealing plate, and an insulative housing configured to secure the tissue sealing plate to the support base. A non-stick coating formed from a liquidphobic structure is disposed on at least a portion of at least one of the opposing jaw members. In one example, the non-stick coating is disposed on at least a portion of each of the tissue sealing plates, the support base, and the insulative housing. In one example, the non-stick coating thickness has a substantially uniform thickness on the tissues sealing plates, the support base, and the insulative housing. In another example, the coating has a non-uniform thickness. In another example, the non-stick coating is discontinuous. In another example, the non-stick coating is continuous.

According to another example of the present disclosure, a method of inhibiting tissue from sticking to an electrically conductive component of an electrosurgical tissue sealing device during application of energy to tissue is provided. The method includes applying a non-stick coating on at least a portion of an electrically conductive component of an electrosurgical tissue sealing device. In one example, the method can include controlling a thickness of the non-stick coating applied to inhibit tissue from sticking to the electrically conductive component during application of energy to the tissue. The thickness of the non-stick coating also allows a sensing of at least one tissue parameter generated via application of energy to the tissue. In another example, the method can include controlling the dimensions of the textured surface. That is, the height, diameter, and spacing of the pillars formed during texturing can be controlled.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

FIG. 1A is a side view of an electrosurgical device having jaws in an open configuration, according to an example of the present application.

FIG. 1B is a perspective view of a bipolar electrode that can be used with the electrosurgical device of FIG. 1A.

FIG. 1C illustrates a close-up view of a surgical needle device.

FIG. 2A is a side view of a portion of an electrosurgical device including the jaws, in accordance with at least one example.

FIG. 2B is a perspective view of the portion of the electrosurgical device in FIG. 2A.

FIG. 3 illustrates an expanded view of a jaw member, in accordance with at least one example.

FIG. 4 illustrates a jaw member including a non-stick coating, in accordance with at least one example.

FIG. 5 illustrates a jaw member including a non-stick coating, in accordance with at least one example.

FIG. 6 illustrates a jaw member including a non-stick coating, in accordance with at least one example.

FIG. 7 illustrates a jaw member including a non-stick coating, in accordance with at least one example.

FIG. 8 illustrates a jaw member including a non-stick coating, in accordance with at least one example.

FIG. 9 illustrates a jaw member including a non-stick coating, in accordance with at least one example.

FIG. 10 illustrates a textured surface, in accordance with at least one example.

FIG 11 illustrates another textured surface, in accordance with at least one example.

FIG. 12 illustrates another textured surface, in accordance with at least one example.

FIG. 13 illustrates an end effector of a surgical cutting device, in accordance with at least one example.

FIG. 14 illustrates a distal portion of a dissecting forceps, in accordance with at least one example.

FIG. 15 is a schematic diagram of a surface on a surgical device coating with a hydrophobic coating, in accordance with at least one example,

FIG. 16 shows a surface with a hydrophobic physical structure on a substrate 1102, in accordance with at least one example.

FIG. 17 shows a laser etched surface that includes hydrophobic physical structure, in accordance with at least one example.

FIG. 18 illustrates an example where a non-stick surface is applied along the perimeter of an electrode, in accordance with at least one example.

FIG. 19 illustrates an example where a non-stick surface is applied along a portion of perimeter of an electrode and along a midsection of the electrode, in accordance with at least one example.

FIG. 20 illustrates an example where a non-stick surface is applied along a portion of perimeter of an electrode, in accordance with at least one example.

FIG. 21 illustrates an example where a non-stick surface is applied to a portion of an overall surface of an electrode, in accordance with at least one example.

FIG. 22 illustrates an example where a non-stick surface is applied to an entire surface of an electrode, in accordance with at least one example.

FIG. 23 illustrates an example where a non-stick surface is applied to an entire surface of a jaw body.

FIG. 24 illustrates an example where a non-stick surface is applied to a portion of a jaw body.

FIG. 25 is a flow chart depicting a method of applying non-stick coating(s) a surgical device.

FIG. 26 is a flow chart depicting a method of applying non-stick coating(s) a surgical device.

In the drawings, which are not necessarily drawn to scale ; like numerals may describe similar components in different views. bike numerals having different letter suffixes may represent different instances of similar components. The drawings illustrate generally, by way of example, but not by way of limitation, various embodiments discussed in the present document.

### DETAILED DESCRIPTION

As described in more detail below with reference to the accompanying figures, the present disclosure is directed to electrosurgical devices having a non-stick coating formed from a liquidphopic structure disposed on one or more components (e.g., tissue sealing plates, jaw members, electrical leads, insulators etc.). The non-stick coating is imparted to the electrosurgical device to provide tissue sticking reduction during tissue sealing.

As used herein, “liquidphobic” or “super-liquidphobic” structures describe, in a general sense, any material that displays anti-liquid properties, e.g., a material that is one or more of hydrophobic (repels water), lipophobic, olephobic and superoleophobic (repels oils and lipids), amphiphobic (a material which is both hydrophobic and lipophobic), hemophobic (repels blood or blood components) or the like. Such materials repel liquids, e.g., by causing the liquid to bead-up on the material's surface and not spread out or wet the material's surface. Thus, as used herein, a substrate that is described as comprising a liquidphobic structure includes substrates that comprise a liquidphobic, super-liquidphobic, hydrophobic, super-hydrophobic, olephobic, super-liquidphobic, amphiphobic and/or super-amphiphobic substrate.

When a drop of a liquid (e.g., water based, lipid based, etc.) rests upon a surface, it will spread out over the surface to a degree based upon such factors as the surface tensions of the liquid and the substrate, the smoothness or roughness of the surface, etc. For example, the liquidphobicity of a substrate can be increased by various coatings that lower the surface energy of the substrate. The quantification of liquidphobicity can be expressed as the degree of contact surface angle (or contact angle) of the drop of the liquid on the surface.

For example, for a surface having a high surface energy (i.e., higher than the surface tension of the liquid drop), a drop of liquid will spread out “wetting” the surface of the substrate. Such surface displays liquidphilicity, as opposed to liquidphobicity. When the surface energy of a substrate is decreased, liquidphobicity is increased (and vice versa). Liquidphobic, including hydrophobic, lipidphobic and/or amphiphobic refer to properties of a substrate which cause a liquid drop on their surface to have a contact angle of 90 degrees (°) or greater. “Super-hydrophobicity,” “super-amphiphobicity,” and “super-liquidphobicity” all refer to properties of substances which cause a liquid drop on their surface to have a contact angle of 150° or greater.

The liquidphobic structures, when applied to electrosurgical devices, can reduce the sticking of tissue during the application of electrosurgical energy for treating tissue. For example, the super-hydrophobicity texture consists of an array of micro pillars that support the water droplets (can be saline or other liquid) and does not adhere to the surface. In contrast, a substrate without the micro-pillars allow the water droplets to spread across the surface.

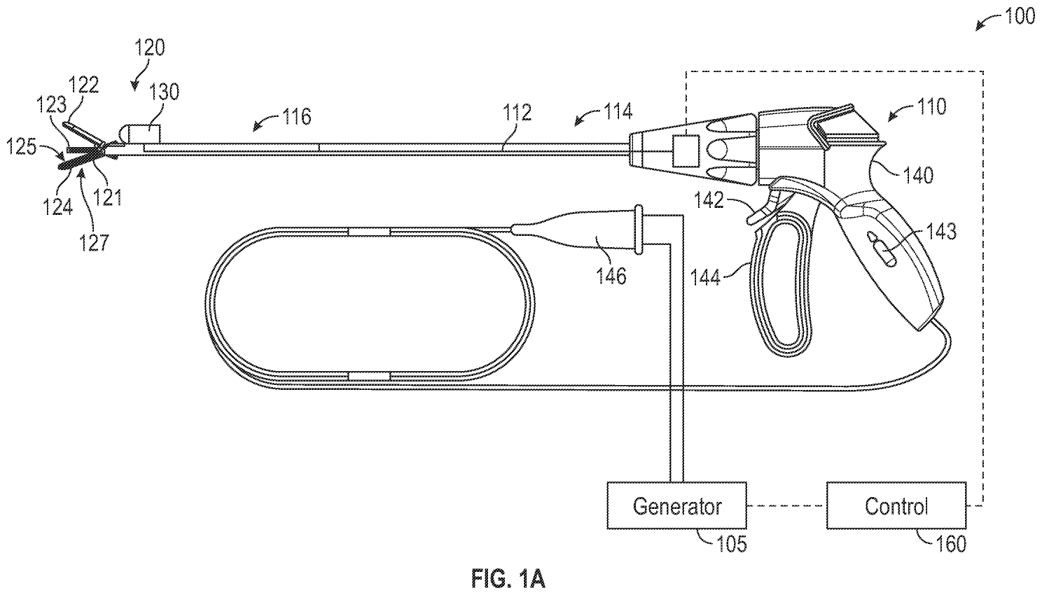

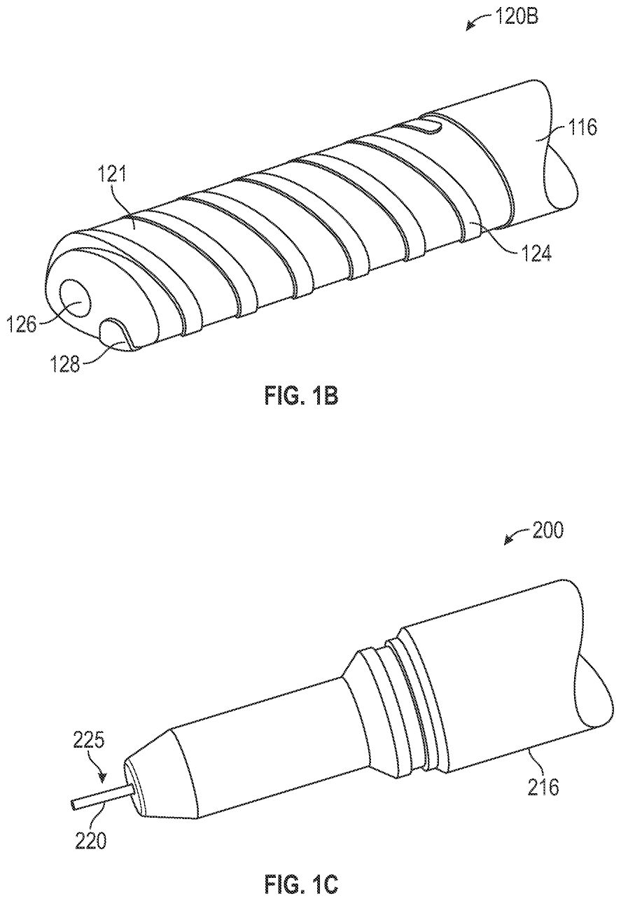

FIG. 1A illustrates a schematic diagram of an example of portions of an electrosurgery system 100, such as can include an electrosurgery device 110 with an electrosurgical end effector 120 such as forceps, spatula, loop or other cutting device, such as containing a blade. As understood the systems or devices described herein can apply to any suitable surgical device such as a forceps, probe, or the like. The device 110 can be connected to an electrosurgical energy generator 105 and a controller 160.

The electrosurgery device 110 can include a longitudinal shaft 112 having a proximal portion 114 and a distal portion 116. The distal portion 116 can include an end effector 120, a jaw body 121 such as which can include a cutting element 123, electrodes 120, and a first and second non-stick layer 125, 127. A proximal portion 114 of the device can be connected to a handpiece 140, such as with actuators 142, 143, and 144. The device 110 can also include a connector 146 such as can be configured to be connected to the generator 105.

The generator 105 can be external to but coupled to the electrosurgical device 110. The generator 105 can provide electrical energy to the end effector 120 of the electrosurgical device 110, such as through the electrical connector 146. The electrical generator 105 can produce a current deliverable by the end effector 120 such as for inducing a coagulation mode of electrosurgery. The electrical generator 105 can be in communication with the controller 160, which can direct the application of electrosurgical energy to the end effector 120 in the electrosurgical device 110.

The type and amount of electrical energy provided by the generator 105 can vary, such as depending on the desired treatment. The electrosurgical waveform produced, the voltage, and the power of the electrosurgical energy being delivered, and the size and surface area of the end effector 120, can affect the depth and the rate of producing heat, which, in turn, can alter the final effect on the target tissue.

The electrosurgery device 110 can include a bipolar or monopolar electrosurgery end effector 120 such as for applying high-frequency alternating polarity electrical current to biological tissue, such as to cut, coagulate, desiccate, or fulgurate the tissue, as may be desired by the surgeon treating the patient.

The electrosurgery device 110 can include a wet field device such as for wet field electrosurgery, such as in a saline solution, or in an open wound. In a wet field device, heating can result from an AC current passing between two electrodes. Heating can be the greatest where the current density is the highest. Thus, smaller surface area electrode can produce a greater amount of heat for treating tissue.

In the device 110, the shaft 112 with the proximal portion 114 and the distal portion 116 can be sized, shaped, or arranged for partial insertion of the device 110 into a patient. The shaft 112 can include or can be made of one or more of a composite, plastic, or metallic material, or other material suitable for surgical applications. The proximal portion 114 can be near an operator, such as a surgeon, when the device 110 is in use. In some cases, the operator can be a robotic arm or other machine. The distal portion 116 can be sized, shaped, or arranged for insertion into the patient so that distal portion 116 is further from the operator during use.

In some cases, the shaft 112 can be sized, shaped, arranged, or otherwise configured for laparoscopy, in some cases, the shaft 112 can be shorter such as for open surgery applications. In some cases, such as for laparoscopy, the shaft can be long. In an open surgery application, the shaft can include a tissue interface element with cutting, coagulating, and sensing elements in or on a distal portion of that device.

Laparoscopy can include, for example, a surgical procedure in which a small incision is made, through which a device is inserted to diagnose or treat conditions. Laparoscopy is considered less invasive than regular open abdominal surgery. In the case of laparoscopy, an optical visualization or imaging device may also be inserted along with the device 110, such as to permit the optical device to allow viewing or imaging such as for the operator to observe the tissue. The optical visualization or imaging device can include a laparoscope, or viewing tube. such as with a camera. In some cases, the optical visualization or imaging device can include an ultrasound type imaging device for the operator to use during treatment.

By contrast, open surgery approaches can involve a larger incision, such as can allow more direct visual observation of cutting of skin and tissue, such to permit the surgeon to have a fuller view of the structures and organs involved in the procedure.

For example, in some applications, the shaft 112 can have a length in a range of 10 mm to 30 mm, inclusive. The shaft 112 can be narrow in a cross-section or a lateral dimension, such as for patient insertion via an incision. For example, the shaft 112 can have a cross-sectional or lateral width in a range of less than 6 mm, inclusive.

The end effector 120 can be located at or near the distal portion 116 of the shaft 112. The end effector 120 can include a bipolar or monopolar electrode and optionally a blade, such as for use in cutting tissue. Bipolar or monopolar electrodes can make use of high frequency electrical current such as to cut, coagulate, desiccate, or fulgurate tissue. With a bipolar electrode configuration, current passes through the tissue between two more closely-spaced electrodes, such as between individual electrode arms of a forceps-type electrode. In a bipolar configuration, the current passes through the tissue between tips of two active electrodes, such as between electrode tips of a bipolar forceps. With a monopolar configuration, current can pass through the tissue between the end effector 120 and a pad on the patient's abdomen or other, separate return electrode. The electrical generator 105 can be connected to both active and return electrodes, such as for sending and receiving current. The end effector 120 can be configured to heat the targeted tissue.

Tissue can see a reduction in resistance as it heats, as the fluid content remains unaltered and does not change to the more resistive state of steam. Thus, during a procedure the risk increases of moving into the undesirable lower resistance range during the heating of some tissues. By increasing the resistance of the devices by about 5 to 10 Ohms, it can have a large impact on a device when connected to a limited source current generator, by essentially moving the power curve towards the lower resistance states.

The handpiece 140 can include one or more user-actuators, such as the actuators, 143, 144. In some cases, these can include one or more of levers, buttons, wheels, switches, triggers, or a combination thereof. One of the actuators 142, 143, 144, can provide a user-interface to control a first switch that selectively connects the end effector 120 to the generator 105 or other circuitry that can provide electrosurgical energy to the first end effector 120 such as for cutting and coagulation. Additional actuators, such as buttons, triggers, or other user-actuatable mechanisms can be included on the handpiece 140 of the device 110 or elsewhere for surgeon use, such as for direction and action of the end effector 120, movement of the shaft 112, or one or more other operations of the device 110.

The electrosurgical device 110, including the triggers on the handpiece 140, the end effector 120, and the one or more sensors 130, can be in communication with the controller 160. The generator 105 can also be in communication with the controller 160.

The controller 160 can include a processor and a memory such as to permit the controller 160 to communicate with and control the generator 105. The controller 160 can be used to allow for both predictive and reactive control of the duty cycle produced by the generator 105.

The controller 160 can operate as a standalone device, or may be networked to other machines. The controller 160 can include a hardware processor, such as a central processing unit (CPU), a graphics processing unit (GPU), a hardware processor core, or any combinations thereof. The controller 160 can further include a memory, including a main memory and a static memory. The controller 160 can include an input device, such as a keyboard, a user interface, and a navigation device such as a mouse or touchscreen.

The controller 160 can additionally include a storage device, a signal generation device, a network interface device, and one or more sensors. The storage device can include a machine readable medium on which is stored one or more sets of data structure or instructions embodying or utilized by any one or more of the techniques described herein. The instructions may also reside, completely or at least partially, within the main memory, within static memory, or within the hardware processor during execution thereof by the controller.

In an example, one or any combination of the hardware processor, the main memory, the static memory, or the storage device may constitute machine readable media, that may include any medium that is capable of storing, encoding, or carrying instructions for execution by the controller 160 and that cause the controller 160 to perform any one or more of the techniques of the present disclosure, or that is capable of storing, encoding or carrying data structures used by or associated with such instructions. Non-limiting machine readable medium examples may include solid-state memories, and optical and magnetic media. The instructions on the controller 160 may further be transmitted or received over a communications network using a transmission medium via a network interface device.

The device 100 in FIG. 1A can be used, for example, to cut tissue, such as a mechanical cutting device assisted with RF (radio frequency) energy derived from the current produced by the generator. For cutting tissue, the generator 105 can produce an electrosurgical energy waveform similar to a sine wave. Cutting can use a continuous electrosurgical energy waveform such as can be able to apply the maximum output power of the generator 105, if desired. By comparison, for coagulating tissue, the peak current output can be greater than for cutting tissue, but with an intermittent or lower duty cycle waveform with lower average power than a cutting waveform. For cutting tissue, the peak voltage can be greater. Cutting tissue can include resection and dissection. Various electrosurgical waveforms can be used for electrosurgical procedures. Rapid heating of tissue using a continuous waveform can result in vaporization, fragmentation, and ejection of tissue fragments, allowing for tissue cutting. Open circuit, voltage of such electrical waveforms can be, for example, from about 300 to about 10,000 V peak-to-peak, inclusive. In some cases, rapid tissue heating can allow for explosive vaporization of interstitial fluid; if the voltage is sufficiently high, such as above 400 V peak-to-peak, the vapor can be ionized, sometimes resulting in conductive plasma allowing flow of electric current from the electrode via the plasma into the tissue.

The generator 105 can be external to but coupled to the electrosurgical device 110. The generator 105 can provide electrical energy to the end effector 120 of the electrosurgical device 110, such as through the electrical connector 146. The electrical generator 105 can produce a current deliverable by the end effector 120 such as for inducing a coagulation mode of electrosurgery. The electrical generator 105 can be in communication with the controller 160, which can direct the application of electrosurgical energy to the end effector 120 in the electrosurgical device 110.

The type and amount of electrical energy provided by the generator 105 can vary, such as depending on the desired treatment. The electrosurgical waveform produced, the voltage, and the power of the electrosurgical energy being delivered, and the size and surface area of the end effector 120, can affect the depth and the rate of producing heat, which, in turn, can alter the final effect on the target tissue.

The electrosurgery device 110 can include a bipolar or monopolar electrosurgery end effector 120 such as for applying high-frequency alternating polarity electrical current to biological tissue, such as to cut, coagulate, desiccate, or fulgurate the tissue, as may be desired by the surgeon treating the patient.

The electrosurgery device 110 can include a wet field device such as for wet field electrosurgery, such as in a saline solution, or in an open wound. In a wet field device, heating can result from an AC current passing between two electrodes. Heating can be the greatest where the current density is the highest. Thus, smaller surface area electrode can produce a greater amount of heat for treating tissue.

In the device 110, the shaft 112 with the proximal portion 114 and the distal portion 116 can be sized, shaped, or arranged for partial insertion of the device 110 into a patient. The shaft 112 can include or can be made of one or more of a composite, plastic, or metallic material, or other material suitable for surgical applications. The proximal portion 114 can be near an operator, such as a surgeon, when the device 110 is in use. In some cases, the operator can be a robotic arm or other machine. The distal portion 116 can be sized, shaped, or arranged for insertion into the patient so that distal portion 116 is further from the operator during use.

In some cases, the shaft 112 can be sized, shaped, arranged, or otherwise configured for laparoscopy, in some cases, the shaft 112 can be shorter such as for open surgery applications. In some cases, such as for laparoscopy, the shaft can be long. In an open surgery application, the shaft can include a tissue interface element with cutting, coagulating, and sensing elements in or on a distal portion of that device.

Laparoscopy can include, for example, a surgical procedure in which a small incision is made, through which a device is inserted to diagnose or treat conditions. Laparoscopy is considered less invasive than regular open abdominal surgery. In the case of laparoscopy, an optical visualization or imaging device may also be inserted along with the device 110, such as to permit the optical device to allow viewing or imaging such as for the operator to observe the tissue. The optical visualization or imaging device can include a laparoscope, or viewing tube, such as with a camera. In some cases, the optical visualization or imaging device can include an ultrasound type imaging device for the operator to use during treatment.

By contrast, open surgery approaches can involve a larger incision, such as can allow more direct visual observation of cutting of skin and tissue, such to permit the surgeon to have a fuller view of the structures and organs involved in the procedure.

For example, in some applications, the shaft 112 can have a length in a range of 10 mm to 30 mm, inclusive. The shaft 112 can be narrow in a cross-section or a lateral dimension, such as for patient insertion via an incision. For example, the shaft 112 can have a cross-sectional or lateral width in a range of less than 6 mm, inclusive.

The end effector 120 can be located at or near the distal portion 116 of the shaft 112. The end effector 120 can include a bipolar or monopolar electrode and optionally a blade, such as for use in cutting tissue. Bipolar or monopolar electrodes can make use of high frequency electrical current such as to cut, coagulate, desiccate, or fulgurate tissue. With a bipolar electrode configuration, current passes through the tissue between two more closely-spaced electrodes, such as between individual electrode arms of a forceps-type electrode. In a bipolar configuration, the current passes through the tissue between tips of two active electrodes, such as between electrode tips of a bipolar forceps. With a monopolar configuration, current can pass through the tissue between the end effector 120 and a pad on the patient's abdomen or other, separate return electrode. The electrical generator 105 can be connected to both active and return electrodes, such as for sending and receiving current. The end effector 120 can be configured to heat the targeted tissue.

Tissue can see a reduction in resistance as it heats, as the fluid content remains unaltered and does not change to the more resistive state of steam. Thus, during a procedure the risk increases of moving into the undesirable lower resistance range during the heating of some tissues. By increasing the resistance of the devices by about 5 to 10 Ohms, it can have a large impact on a device when connected to a limited source current generator, by essentially moving the power curve towards the lower resistance states.

The handpiece 140 can include one or more user-actuators, such as the actuators, 143, 144. In some cases, these can include one or more of levers, buttons, wheels, switches, triggers, or a combination thereof. One of the actuators 142, 143, 144, can provide a user-interface to control a first switch that selectively connects the end effector 120 to the generator 105 or other circuitry that can provide electrosurgical energy to the first end effector 120 such as for cutting and coagulation. Additional actuators, such as buttons, triggers, or other user-actuatable mechanisms can be included on the handpiece 140 of the device 110 or elsewhere for surgeon use, such as for direction and action of the end effector 120, movement of the shaft 112, or one or more other operations of the device 110.

The electrosurgical device 110, including the triggers on the handpiece 140, the end effector 120, and the one or more sensors 130, can be in communication with the controller 160. The generator 105 can also be in communication with the controller 160.

The controller 160 can include a processor and a memory such as to permit the controller 160 to communicate with and control the generator 105. The controller 160 can be used to allow for both predictive and reactive control of the duty cycle produced by the generator 105.

The controller 160 can operate as a standalone device, or may be networked to other machines. The controller 160 can include a hardware processor, such as a central processing unit (CPU), a graphics processing unit (GPU), a hardware processor core, or any combinations thereof. The controller 160 can further include a memory, including a main memory and a static memory. The controller 160 can include an input device, such as a keyboard, a user interface, and a navigation device such as a mouse or touchscreen.

The controller 160 can additionally include a storage device, a signal generation device, a network interface device, and one or more sensors. The storage device can include a machine readable medium on which is stored one or more sets of data structure or instructions embodying or utilized by any one or more of the techniques described herein. The instructions may also reside, completely or at least partially, within the main memory, within static memory, or within the hardware processor during execution thereof by the controller.

In an example, one or any combination of the hardware processor, the main memory, the static memory, or the storage device may constitute machine readable media, that may include any medium that is capable of storing, encoding, or carrying instructions for execution by the controller 160 and that cause the controller 160 to perform any one or more of the techniques of the present disclosure, or that is capable of storing, encoding or carrying data structures used by or associated with such instructions. Non-limiting machine readable medium examples may include solid-state memories, and optical and magnetic media. The instructions on the controller 160 may further be transmitted or received over a communications network using a transmission medium via a network interface device.

The device 100 in FIG. 1A can be used, for example, to cut tissue, such as a mechanical cutting device assisted with RF (radio frequency) energy derived from the current produced by the generator. For cutting tissue, the generator 105 can produce an electrosurgical energy waveform similar to a sine wave. Cutting can use a continuous electrosurgical energy waveform such as can be able to apply the maximum output power of the generator 105, if desired. By comparison, for coagulating tissue, the peak current output can be greater than for cutting tissue, but with an intermittent or lower duty cycle waveform with lower average power than a cutting waveform. For cutting tissue, the peak voltage can be greater.

Cutting tissue can include resection and dissection. Various electrosurgical waveforms can be used for electrosurgical procedures. Rapid heating of tissue using a continuous waveform can result in vaporization, fragmentation, and ejection of tissue fragments, allowing for tissue cutting. Open circuit voltage of such electrical waveforms can be, for example, from about 300 to about 10,000 V peak-to-peak, inclusive. In some cases, rapid tissue heating can allow for explosive vaporization of interstitial fluid; if the voltage is sufficiently high, such as above 400 V peak-to-peak, the vapor can be ionized, sometimes resulting in conductive plasma allowing flow of electric current from the electrode via the plasma into the tissue.

The bipolar electrode 120B is shown in greater detail in FIG. 1B. The electrode 120B can extend distally from the shaft. The electrode 120B can include a ceramic probe tip 121 and electrode material 124, along with coating 125. The electrode 120B can be used, for example, for surgery such as colon surgery or intestinal surgery. The electrode 120B can be used to address polyps or bleeders in the patient. For example, where a surgeon removes a polyp in patient tissue, the bipolar electrode 120B can be used to stop bleeding at the cut site by coagulating the tissue. However, if the electrode 120B were to stick to the tissue being treated, a risk of a larger bleed is possible if the tissue tears during operation. To address this, the electrode 120B includes the non-stick coating 125.

The ceramic probe tip 121 can be any suitable ceramic material for hosting the electrode material 124. The electrode material 124 can serve as the electrical conductors along the probe tip 122. The electrode material 124 can be printed, etched, or adhered to the ceramic probe tip 121. In some cases, the electrode material 124 can be in a pattern, such as in a spiral pattern.

The electrode material 124 can include various loops, where the first loop can be positive, and the second loop can be negative, along the length of the ceramic probe tip 121. At varying points in time, as the provided radio frequency (RF) from the generator switches during the procedure, energy can be passed from one loop to the next along the length of the electrode material 124, to allow for treatment of the targeted tissue. In an example, the electrode material 124 can be a conductive metallic or alloy material situated on the ceramic probe tip 121.

FIG. 1C illustrates a close-up view of a surgical needle device. In some cases, instead of a bipolar electrode such as electrode 120 shown and discussed above, a non-stick coating can be applied to a surgical needle device 200. The device 200 can include needle 220 with coating 225, and shaft 230 extending proximally from the needle 220.

The needle 220 can be size, shape, or arranged, for insertion into tissue. The needle 220 can be configured to pass energy into the tissue. The device 200 can be in a monopolar configuration, where a single needle is used with a pad, or in a bipolar configuration, where two needles are used. The device 200 can be used, for example, to treat a liver tumor, kidney tumor, or tissue in the lungs. In this case, the electrically conductive needle is inserted into the tissue, and used to pass energy therethrough. This can produce heat in and around the treatment area for surgical treatment, such as for coagulation. In some cases, the puncture site can bleed, or tissue tearing can occur if a portion of the needle sticks to the targeted tissue.

The needle 220 can include a non-stick coating 225 to prevent tissue sticking. The electrically conductive portion of the needle 220 can be coated with such a coating 225. As discussed above, the non-stick coating 225 can be a polymer based coating, an etched coating, a hydrophobic coating, or combinations thereof.

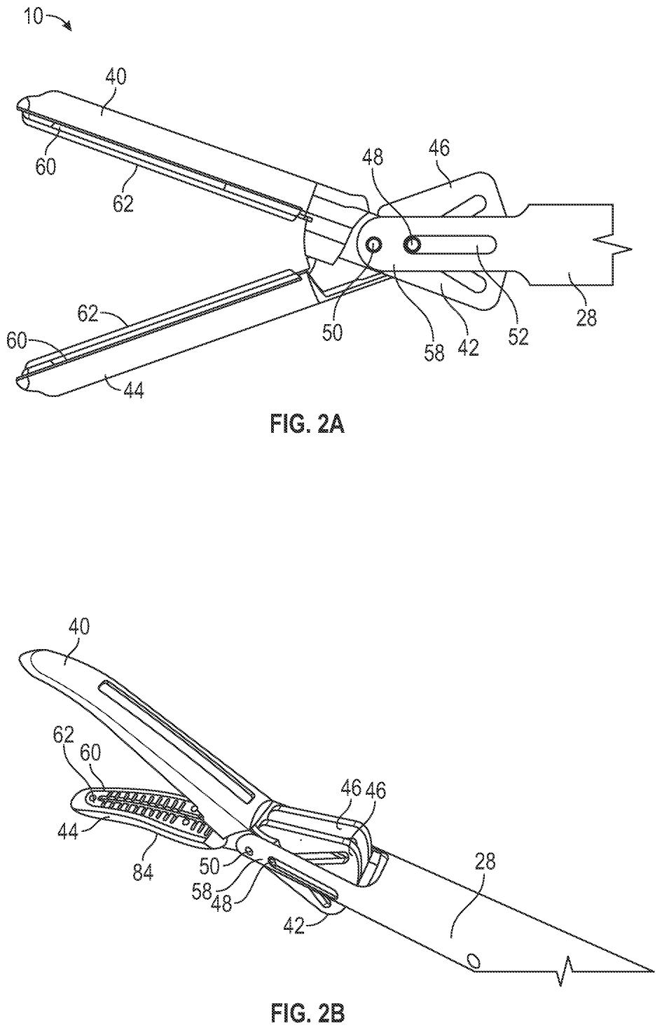

FIG. 2A illustrates a side view of a portion of forceps 10, in accordance with at least one example of this disclosure. FIG. 2B illustrates a perspective view of a portion of the forceps 10. FIGS. 2A and 2B are discussed below concurrently. The forceps 10 can include a top jaw 40 (including flanges 42), a bottom jaw 44 (including flanges 46), a drive pin 48, a pivot pin 50, an inner shaft 52, and an outer shaft 28. The outer shaft 28 can include outer arms 58.

The forceps 10 can be used with various surgical procedures. The end effector 16 includes pair of opposing jaw members 40, 44 that rotate about a pivot pin 50 and that are movable relative to one another to grasp tissue. As seen in FIGS. 2A and 2B, the jaw members 40, 44 include an electrically conductive sealing plate

In one example, a sensor can be disposed on or proximate to at least one of the jaw members 40, 44 of the forceps 10 for sensing tissue parameters (e.g., temperature, impedance, etc.) generated by the application of electrosurgical energy to tissue via the jaw members 40, 44. The sensor may include a temperature sensor, tissue hydration sensor, impedance sensor, optical clarity sensor, or the like. A cable, coupling the forceps 10 to an electrosurgical generator, can transmit sensed tissue parameters as data to the electrosurgical generator having suitable data processing components (e.g., microcontroller, memory, sensor circuitry, etc.) for controlling delivery of electrosurgical energy to the forceps 10 based on data received from the sensor.

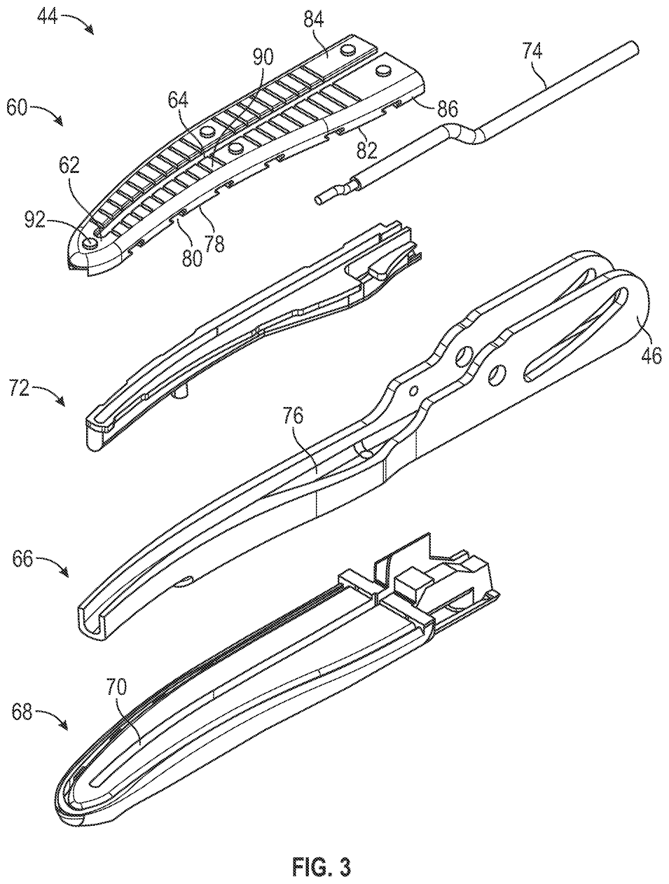

FIG. 3. illustrates an exploded view of the jaw member 44, as shown in FIGS. 2A and 2B. Jaw member 44 can be identical to jaw member 40. The jaw member 44 can include the electrically conductive sealing plate 60, e.g., an electrode, including a blade slot 64, a frame 66 (including flanges 46), an overmold 68 including a blade slot 70, and a support 72. A wire 74 can electrically couple the electrically conductive sealing plate 60 to an energy source.

The overmold 68 can include the blade slot 70, which can be aligned with the blade slot 64 of the electrically conductive sealing plate 60 when the overmold 68 is secured to the electrically conductive sealing plate 60 (such as when the overmold 68 is overmolded to the frame 66 and the electrically conductive sealing plate 60. In an example, the frame 66 can include a slot 76 that can receive the support 72 therein. The support 72 can help to support the electrically conductive sealing plate 60 on the frame 66.

The electrically conductive sealing plate 60 includes an underside surface 82 that can include an electrically insulative layer 86 bonded thereto or otherwise disposed thereon. The electrically insulative layer 86 can electrically insulate the electrically conductive sealing plate 60, from the support 72 and the frame 66. In one example, the electrically insulative layer 86 is formed from polyimide. However, in other examples, any suitable electrically insulative material may be utilized, such as polycarbonate, polyethylene, etc.

Additionally, the jaw member 44 include an external surface 84 that includes a non-stick coating 62 disposed thereon. The non-stick coating 62 may be disposed on selective portions of either of the jaw members 40, 44, or may be disposed on the entire external surface 84. In some examples, the non-stick coating 62 is disposed on a tissue-engaging surface 90 of the electrically conductive sealing plate 60. The non-stick coating 62 is configured to reduce the sticking of tissue to the electrical conducting sealing plates, the jaw members, the electrical leads, and/or the surrounding insulating material.

The support 72 is configured to support the electrically conductive sealing plate 60 thereon. The electrically conductive sealing plate 60 may be affixed atop the support 72 that can be coupled to or integral with the frame 66. The electrically conductive sealing plate 60 can be coupled to the support 72 and/or frame 66, by any suitable method including but not limited to snap-fitting, overmolding, stamping, ultrasonic welding, laser welding, etc. The support 72, frame 66, and the electrically conductive sealing plate 60 is at least partially encapsulated by overmold 68, by way of an overmolding process to secure the electrically conductive sealing plates 60 to the support 72 and the frame 66.

The electrically conductive sealing plate 60 can include teeth 78 that can define recesses 80. The recesses 80 can be located on a side edge of the electrically conductive sealing plate 60. The recesses 80 can be configured to let material of the overmold 68 infiltrate (or fill in) the recesses (or spaces or gaps) 80 so that the electrically conductive sealing plate 60 is secured to the overmold 68. In an example, the electrically conductive sealing plate 60 is an electrode (or can include an electrode) which can be electrically connected to the wire (or conduit) 74.

The electrically conductive sealing plate 60 is coupled to wire 74 (e.g., electrical lead/conduit), via any suitable method (e.g., ultrasonic welding, crimping, soldering, etc.). The wire 74 serves to deliver electrosurgical energy (e.g., from an electrosurgical energy generator) to the electrically conductive sealing plate 60.

Jaw member 44 may also include a series of stop members 92 disposed on the tissue-engaging surface of the electrically conductive sealing plate 60 to facilitate gripping and manipulation of tissue and to define a gap between the jaw members 40, 44 during sealing and cutting of tissue. The series of stop members 92 may be disposed (e.g., formed, deposited, sprayed, affixed, coupled, etc.) onto the electrically conductive sealing plate 60 during manufacturing. Some or all of the stop members 92 may be coated with the non-stick coating 62 or, alternatively, may be disposed on top of the non-stick coating 62.

As discussed herein, the non-stick coating is applied to portions of the electrosurgical device to provide tissue adherence resistant (anti-stick) properties. Any structure or material capable of providing the desired functionality (namely, reduction of tissue sticking while simultaneously maintaining sufficient electrical transmission to permit tissue sealing) may be used as the non-stick coating, provided it has adequate biocompatibility. In some examples, the material may be porous to allow for electrical transmission.

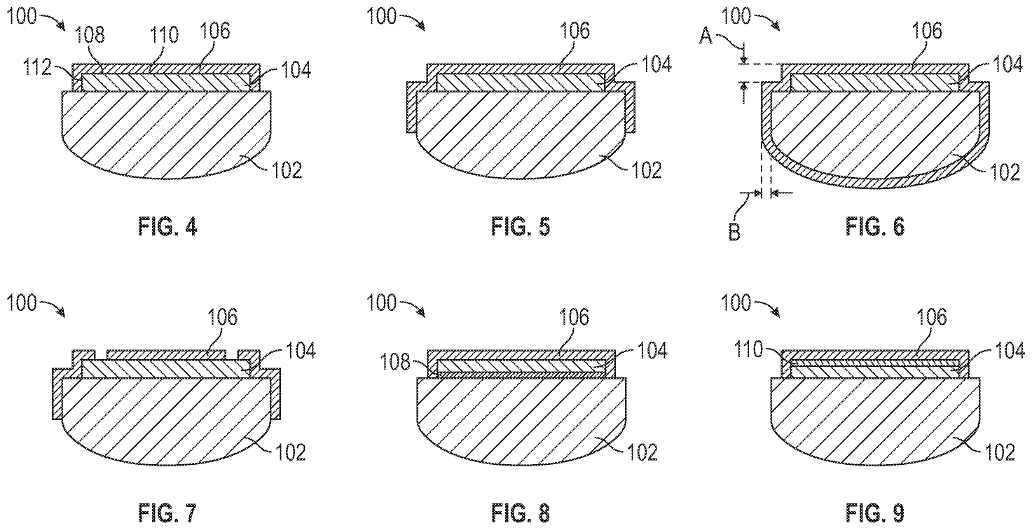

FIGS. 4 through 9 provide simplified examples of a jaw member 400 including a body portion 402 and an electrically conductive sealing plate 404 (hereinafter “sealing plate 404”) having a non-stick coating 406. The examples shown can be applied to jaw members 40, 44 discussed herein. Further, any combination of FIGS. 5 through 9 can be used to apply the non-stick coating to jaw members.

As discussed herein, the non-stick coating can be a liquidphobic structure formed from a coating applied directly to portions of the electrosurgical device. The thickness and the location of the non-stick coating can vary. In some embodiments, the thickness of the non-stick coating can vary such that the non-stick coating is non-uniform.

FIG. 4 illustrates an example where the non-stick coating 406 is applied to cover the entire external surface 408 of the sealing plate 404 including the tissue engaging surface 410 and the side surface 412 of the sealing plate 404. In one example, the coating thickness can be uniform or not uniform.

FIG. 5 illustrates an example where the non-stick coating 406 is applied to cover the entire external surface 408 of the sealing plate 404 including the tissue engaging surface 410 and the side surface 412 of the sealing plate 404. However, compared to the example in FIG. 4, the non-stick coating 406 extends past the sealing plate 404 and onto the body portion 402 of the jaw member 400. In one example, the coating thickness can be uniform or not uniform along the tissue engaging surface 410 and the body portion 102.

FIG. 6 illustrates an example where the non-stick coating 406 is applied to cover the entire external surface 408 of the sealing plate 404 including the tissue engaging surface 410 and the side surface 412 of the sealing plate 404. However, the non-stick coating 406 extends past the sealing plate 404 and onto the entire body portion 402 of the jaw member 400. As illustrated in FIG. 6, a first portion of the non-stick coating can have a thickness A. The non-stick coating along a majority of the tissue engaging surface 410 of the electrically conductive sealing plates can have the non-stick coating thickness A, and portions of the non-stick coating not on the tissue engaging surface of the electrically conductive sealing plates can have thickness 13, which is different from thickness A. In one example, thickness A can be less than thickness B. In another example thickness A can be greater than thickness B. Thus, the example shown in FIG. 6 illustrates the sealing plate 440 having a different thickness compared to the thickness of the coating on the jaw body 402.

FIG. 7 illustrates an example where the non-stick coating 406 is applied to cover a portion of the external surface 408 of the sealing plate 404 and a portion of the jaw body 402. For example, the non-stick coating along the sealing plate 404 can include portions that do not include any non-stick coating, i.e., discontinuous.

FIG. 8 illustrates an example similar to the example shown in FIG. 5 but an insulative layer 408 is disposed between the sealing plate 404 and the jaw body 402. The insulative layer can be formed from polyimide. However, in other examples, any suitable electrically insulative material may be utilized, such as polycarbonate, polyethylene, etc.

FIG. 9 illustrates an example similar to the example shown in FIG. 5 but a first coating 406 is deposited between the sealing plate 404 and the non-stick coating 406. The first coating 406 can be a material that can increase the hardness and/or durability of the device. In one example, the first coating 406 can be formed from one of chromium nitride and titanium nitride. In one example, the thickness of the TiN or the CrN coating can be around 150 microns. While, e.g., the TiN is a slight electrical insulator compared to the steel of the sealing plate, it is negligible and would have little effect on the overall resistance with the first coating 406 formed from a siloxane coating.

Various coatings are contemplated. In one example, the liquidphobic structure can be selected from at least one of tungsten disulfide (WS2), siloxanes such as hexamethyldisiloxane (HMDSO), polydimethylsiloxane (PDMSO), and tetramethyldisiloxane (TMDSO), fluorosilane containing compounds, glass, perfluoropolyether derivates, nanotube coatings, and other hydrophobic and superhydrophobic coatings.

Examples of when WS2 is deposited is shown and described in U.S. Pat. No. 6,966,909, the entire contents of which are incorporated herein by reference.

In one example when siloxanes are used, the siloxanes can be deposited via plasma enhanced chemical vapor deposition. The application can be controlled to form a coating having a thickness within a range of about 10 nanometers (nm) to about 250 nm. In one example, the thickness is in a range of about 10 nm to about 30 nm. In one example, the thickness is in a range of about 90 nm to about 250 nm.

As discussed herein, alternatively or in combination with the coatings described herein, the surfaces of the electrosurgical device can be textured to provide a textured surface. The textured surface forms the liquidphobic structure that can provide non-stick properties. Liquidphobic structures for use in the practice of the present invention include one of texturing a substrate to include at least one of a micro-topography and a nano-topography. In one example, the textured portion can further include a coating as described herein that is applied to the textured surface.

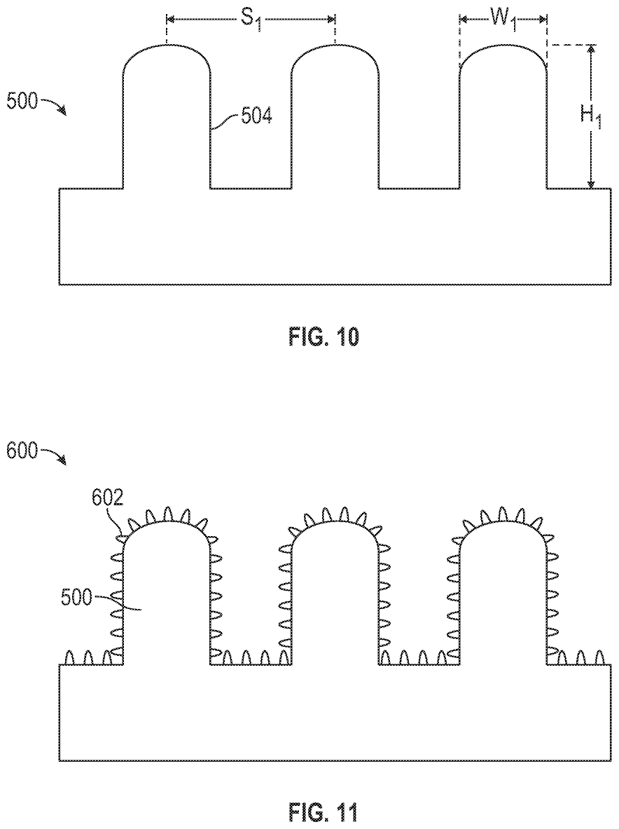

In one example, the textured surface can include a micro-topography, such as micro-pillars. As seen in FIG. 10, the substrate 500, e.g., the electrically conductive tissue sealing plates, can include micro-pillars 504. The micro-pillars 504 can have a height H1 within a range of about 10 microns to about 20 microns, a width W1 within a range of about 70 microns to about 150 microns, and a center-to-center spacing S1 of about 120 microns to about 200 microns.

As discussed herein, the micro-pillars 504 can be formed while the substrate is being formed, e.g., during molding, or can be formed onto the substrate after manufacturing as a post-treatment. Post-treatment micro-topography formation can include, but is not limited to, laser etching, chemical etching, and micro machining. The method selected can depend on the type of material of the substrate. For example, for the electrically conductive tissue sealing plates, generally formed from stainless steel, the micro-topography formation can include the laser etching, chemical etching, and micromachining. In an example where the substrate is a polymer, the micro-topography formation can be done while the substrate is being molded into shape.

In one example, the textured surface can be a topographically complex surface including two “layers” of surfaces having distinct and varied scale ranges. The topographically complex surface generally includes a submicron (nanostructure) surface being superimposed onto the micro-scale roughened surface. “Micro-scale,” as used herein, should be understood to describe an article or feature generally measured in microns such as, for example, 1 micron to 100 microns. “Submicron” or “nanoscale,” as used herein, should be understood to describe an article or feature generally measured in nanometers such as, for example, 1 nanometer to 500 nanometers.

As illustrated in FIG. 11, the substrate 600 includes the micro-pillars 604, as discussed herein, but includes nanostructures 602 superimposed onto the micro-pillars 604. The nanostructures 602 can be nano-pillars and can be formed by spraying, dipping, plasma deposition.

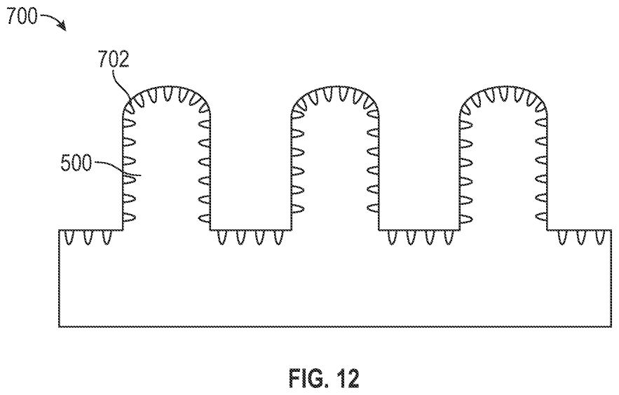

As illustrated in FIG. 12, the substrate 700 includes the micro-pillars 704, as discussed herein, but includes nanostructures 702 superimposed onto the micro-pillars 704. As compared to the nanostructures in FIG. 11 that are formed on the surface of the micro-pillars 703, the nanostructures 702 in FIG. 12 are formed into the micro-pillars 704 and are tube-like structures. In one example, the nanostructures 702 can be formed form potentiostatic anodization.

Additionally, the substrates shown in FIGS. 10-12, can be combined with the coating discussed herein to form the liquidphobic structure.

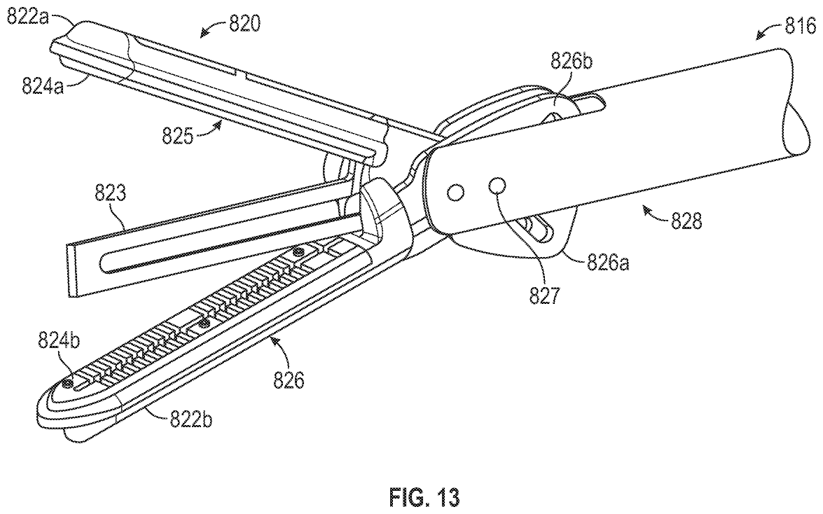

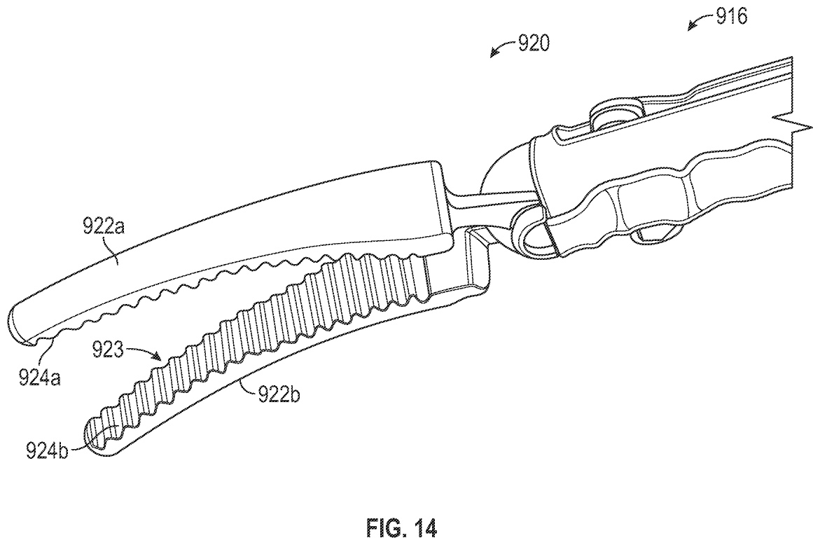

Shown in FIG. 13, the end effector 820 can be a surgical cutting device, such as surgical forceps with a central blade, or other RF type cutting device, such as the examples discussed below with reference to FIGS. 13 and 14.

FIGS. 13 and 14 illustrate various exemplary electrosurgical devices that can contain the non-stick surfaces disclosed herein. Each of the cutting elements in these devices can have a non-stick layer to prevent build-up of tissue.

FIG. 13 illustrates a distal portion 816 of a vessel sealing forceps 820 with non-stick layers 825, 826. The surgical jaw 820 can include jaws 822a, 822b, electrode plates 824a, 824b, cutting element 823, non-stick layer 825, flanges 826a, 826b, pivot point 827, and channel 828.

In vessel sealing forceps 820, the jaws 822a, 822b, can be hinged opposite each other and actuatable via one or more controls on the handpiece. The user can open and close the jaws 822a, 822b, as desired during surgery. The electrode plates 824a, 824b can be on either jaw 822a, 822b, to allow application of current from the generator to the target tissue, such as for vessel sealing. For example, during operation, the surgeon can close the jaws 822a, 822b, around the target tissue, and activate current flow to the electrode plates 824a, 824b, which can seal the tissue. The forceps 820 jaws 822a can be articulated, for example, through movement of the flanges 826a, 826b, around the pivot point 827.

The cutting element 823 can be configured to move in and out of a channel 828 in the body of the distal portion 816 of the device. When the forceps jaws 822a, 822b, have been used to seal tissue, the surgeon can activate extension of the cutting element 823 outward between the jaws 822a, 822b. The cutting element 823 can extend through the channel and cut the target tissue.

The non-stick layer 825 can be on at least a portion of the electrode plates 824a, 824b. In some cases, the non-stick layer 825 can be in and around the cutting channel 828 to prevent tissue build-up in the channel 828 when the cutting element 823 is moved in and out of the channel.

FIG. 14 illustrates a distal portion 916 of a dissecting forceps 920 with a cutting element 923 and a non-stick layer 925 in an example. The dissecting forceps 920 can include curved jaws 922a, 922b, electrode plates 924a, 924b, and non-stick layers 925, 927. In dissecting forceps 920, the jaws themselves can act as the cutting element.

The dissecting forceps 920 can be similar to the forceps discussed above, but can be used for dissection of tissue. The jaws 922a, 922b, can be curved to allow for surgeon articulation and grasping of tissue. This can allow for secure grasping, dissecting, retracting, and coagulating of tissue. The plates 924a, 924b, can be serrated to allow for these actions. The plates 924a, 924b, can be fully or partially coated with the non-stick layer 925. In some cases, other types of forceps, such as a cutting forceps, can alternatively be used. In this case, the cutting portion of the forceps would be at least partially coated with the non-stick layer 925.

Referring back to FIG. 1, the end effector 120 can include a first non-stick layer 125 applied to the electrode plates and, optionally, include a second non-stick layer 127 applied to the polymeric body portion of the jaws. As discussed herein, the first non-stick layer 125 can be applied to the tissue sealing surfaces, e.g., such as an electrode surface. The second non-stick layer 127 can be applied to the polymeric body of the jaw. The first non-stick layer 125 can have a surface adherence less than that of the electrodes and the second non-stick layer 125 can have a surface adherence less than that of the body of the jaw members.

In an example, the first non-stick layer 125 can include an etched coating including one or more hydrophobic pillars or superhydrophobic pillars superimposed on the electrodes 120. With an etched layer 125, a nanostructure of hydrophobic pillars can act as a superhydrophobic coating with a low surface energy, reducing sticking. The etched layer 125 can be in any suitable pattern for the non-stick coating to reduce or prevent tissue sticking. The etched layer 125 can be applied, for example, by printing, chemical etching, laser etching, chemical bombardment, or other suitable techniques. Such examples for providing non-stick structures are described in U.S. Provisional Patent Application Ser. No. 63/143,353, which is incorporated by reference herein in its entirety.

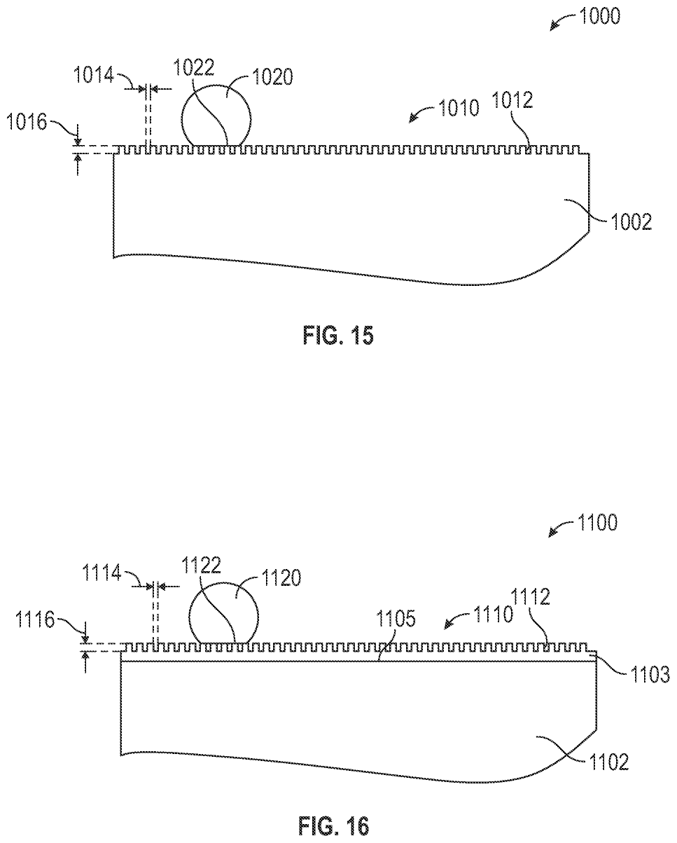

In some cases, it may be beneficial to have different hydrophobic physical structures on different surfaces of components of the device. The hydrophobic physical structure may be on all or a portion of a surface of the device 110, and different hydrophobic physical structures may be used on different surfaces or components of a device. Example hydrophobic structures are discussed below in FIGS. 15-17.

FIG. 15 is a schematic diagram of a surface on a surgical device coating with a hydrophobic coating in an example. FIG. 15 shows one example of a surface with a hydrophobic physical structure 1010 on a substrate 1002 (e.g., an electrode surface). As discussed in examples above, the hydrophobic physical structure 1010 may be on all or a portion of a surface, and different hydrophobic physical structures 1010 may be used on different surfaces or components of an electrosurgical cutting device. The hydrophobic physical structure 1010 may be on only a portion the end effector.

As shown in FIG. 15, in one example, the hydrophobic physical structure 1010 includes asperities 1012 having a height 1016 and a pitch 1014. The hydrophobic physical structure 1010 can be described by the following equation:

$${Λ}_{C} = \frac{- ρ {{gV}^{1 / 3} ( ( \frac{1 - cos ( {θ}_{a} )}{sin ( {θ}_{a} )} ) ( 3 + {( \frac{1 - cos ( {θ}_{a} )}{sin ( {θ}_{a} )} )}^{2} ) )}^{2 / 3}}{{( 36 π )}^{1 / 3} γ cos ( {θ}_{a , 0} + w - 90 )}$$

where Λ is a contact line density, and Λ_{c} is a critical contact line density; ρ=density of the liquid droplet; g=acceleration due to gravity; V=volume of the liquid droplet; θₐ=advancing apparent contact angle; θ_{a,0}=advancing contact angle of a smooth substrate; γΔsurface tension of the liquid; and w=tower wall angle.

The contact line density Λ is defined as a total perimeter of asperities over a given unit area.

In one example, if Λ>Λ_{c} then a droplet 1020 of liquid are suspended in a Cassie-Baxter state. Otherwise, the droplet 1020 will collapse into a Wenzel state. In one example when a Cassie-Baxter state is formed, an ultra-hydrophobic condition exists, and a low adhesion surface is formed. FIG. 15 illustrates a Cassie-Baxter state, where the droplet 1020 rests on top of the asperities 1012 at interface 1022. Although rectangular asperities are shown for illustration purposes, the invention is not so limited. Asperity shapes are taken into account in the formula above, at least in the tower wall angle (w) term.

In the example of FIG. 15, the asperities are formed directly from a bulk material, and are not formed from a separate coating. One method of forming asperities directly from a bulk material includes chemical etching. Another example of forming asperities directly from a bulk material includes laser etching or ablation. Another example of forming asperities directly from a bulk material includes ion etching.

FIG. 16 shows another example of a surface with a hydrophobic physical structure 1110 on a substrate 1102. As discussed in examples above, the hydrophobic physical structure 1110 may be on all or a portion of a surface, and different hydrophobic physical structures 1110 may be used on different surfaces or components of an endoscope. For example, the hydrophobic physical structure 1110 may be on an entire outer surface of an end effector. The hydrophobic physical structure 1110 may be on only a portion of an outer surface of an end effector.

As shown in FIG. 16, in one example, the hydrophobic physical structure 1110 includes asperities 1112 having a height 1116 and a pitch 1114. However, in the example of FIG. 16, the hydrophobic physical structure 1110 is formed as part of a coating 1103 that forms a direct interface 1105 with substrate 1102. FIG. 16 illustrates a Cassie-Baxter state, where the droplet 1120 rests on top of the asperities 1112 at interface 1122.

In one example, the asperities 1112 are formed by application of nanoparticles to a surface of the substrate 1102 to form the coating 1103. In one example, the asperities 1112 are formed by application of nanoparticles to a surface of the coating 1103. In one example, the nanoparticles include hexamethyldisiloxane (HMDSO) particles. In one example, the nanoparticles include tetramethyldisiloxane (TMDSO) particles. In one example, the nanoparticles include fluorosilane particles. Other nanoparticle materials are also within the scope of the invention. In one example, a hydrophobic chemistry of the nanoparticle, in combination with a nano scale asperity structure as shown in FIG. 16 provide better hydrophobicity compared to a hydrophobic chemistry alone.

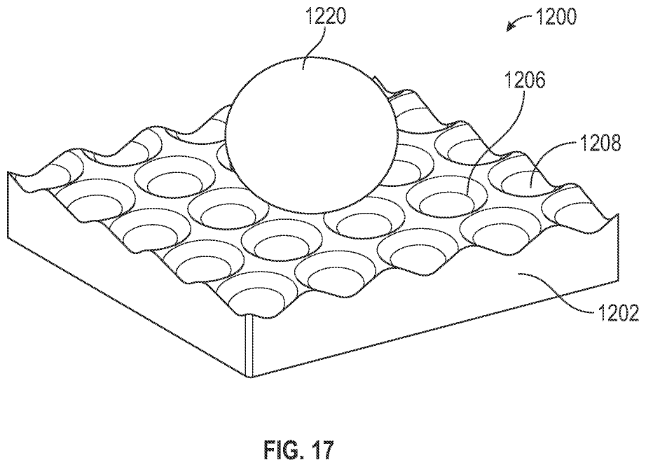

FIG. 17 shows one example of a laser etched surface 1200 that includes hydrophobic physical structure as described above. In the example of FIG. 17, a gaussian hole array is formed by applying laser energy to a surface of a substrate 1202 in a controlled regular pattern to form holes 1206. A shape of the holes 1206 is characterized as gaussian due to the energy distribution of laser energy in forming the array. In the example shown, a number of asperities 1208 are formed in the process that may be spaced and arranged in an array that provides a Cassie-Baxter state as described above. A liquid droplet 1220 is illustrated on the hydrophobic physical structure similar to the droplet from FIG. 15, or the droplet from FIG. 16.

In some cases, the second non-stick layer 127 can be a coating, such as polydimethylsiloxane, hexadimethylsiloxane, or tetramethyldisiloxane. In an example, the non-stick layer 127 can have a thickness in range of about 10 nm to about 300 nm. As discussed herein, because the second non-stick coating 127 is applied to the external surface of the jaw body, there are no concerns regarding thick coatings on electrodes that would prevent application of energy or sensing capabilities. In some cases, the non-stick layer 127 can have a substantially uniform thickness. In some cases, the non-stick layer 127 can have a non-uniform thickness. In some cases, the non-stick layer 127 can be discontinuous. In some cases, the non-stick layer can be continuous. The non-stick layer 127 can include one or more asperities, such as nanoparticles. The non-stick layer 127 can include an electrically insulated or a non-conductive material. In some cases, the non-stick layer 127 can include a hydrophobic surface structure, a coating, or a combination thereof. In some cases, the non-stick layer 127 can overlap a portion of the electrode 120.

In some cases, the layer 127 can include a polymeric-based coating, such as a fluoropolymer type coating. In some cases, the layer 127 can include a Polytetrafluoroethylene (PTFE) coating. In some cases, the layer 127 can include a polysiloxane or a fluorosilane coating. For example, materials such as silicone and silicone resins can be used for the non-stick coating. In one example, the silicone and silicone resins can be applied using a plasma deposition process to precisely control thickness, and can withstand the heat generated during tissue sealing. Silicone resins suitable for the non-stick coating include, but are not limited to, polydimethyl siloxanes, polyester-modified methylphenyl polysiloxanes, such as polymethylsilane and polymethylsiloxane, and hydroxyl functional silicone resins. In some examples, the non-stick coating is made from a composition including a siloxane, which may include hexamethyldisiloxane, tetramethylsilane, hexamethyldisilazane, or combinations thereof.

In an example, the non-stick coating is a polydimethylsiloxane (“PMDSO” coating. In one example, the non-stick coating is a hexamethyldisiloxane (“HMDSO”) coating. In another example, the non-stick coating is a tetramethyldisiloxane (TMDSO or TMDS). In some cases, the layer 127 can include a thin layer of hexamethyldisiloxane (HMDSO), of a thickness of a few nano meters. HMDSO is electrically resistive, but the thinness of the coating can allow passage of RF energy therethrough.

The application of the non-stick coating 127 may be accomplished using any system and process capable of precisely controlling the thickness of the coating. In some examples, HMDSO is deposited on the electrically conductive sealing plates using plasma enhanced chemical vapor deposition (PECVD) or other suitable methods such as atmospheric pressure plasma enhanced chemical vapor deposition (AP-PECVD). For example, the application of the polydimethylsiloxane coating may be accomplished using a system and process that includes a plasma device coupled to a power source, a source of liquid and/or gas ionizable media (e.g., oxygen), a pump, and a vacuum chamber. The power source may include any suitable components for delivering power or matching impedance to the plasma device. More particularly, the power source may be any radio frequency generator or other suitable power source capable of producing electrical power to ignite and sustain the ionizable media to generate a plasma effluent. Application of the coating is discussed in more detail below with reference to FIG. 9.

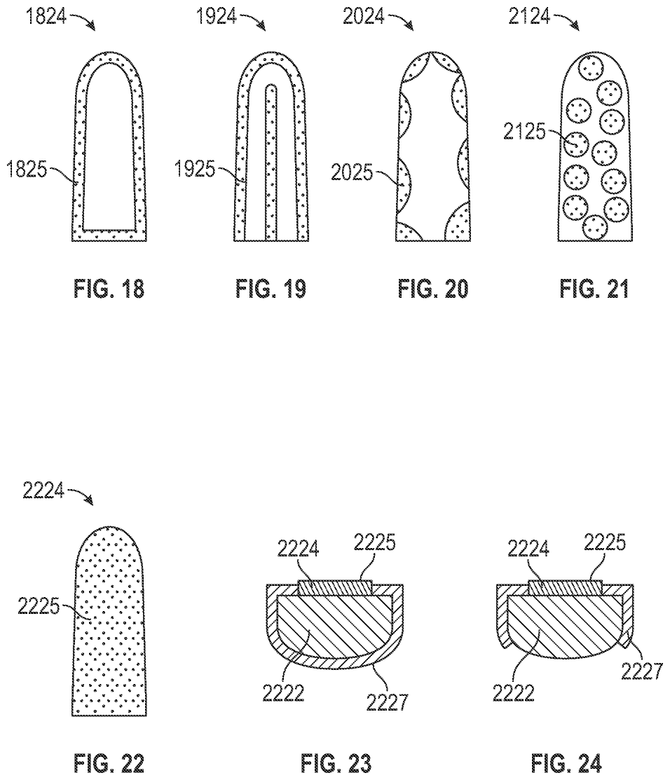

As discussed above, the application and location of the non-stick coating 125 can vary. The present inventors have determined that localized laser texturing along various portions of the electrode 224 can provide advantages. Depending on the device and surgical use, various portions of the electrode 224 can be targeted with laser texturing. Hydrophobic or superhydrophobic surface structures formed by the surface texturing can provide a mechanically robust and wear resistant non-stick surface, as compared to non-stick coatings, e.g., polysiloxanes. Additionally, by forming the non-stick layer with non-stick surface structures eliminates the issues associated with non-stick coatings regarding diminishing the electrical and/or sensing capabilities of the electrode. FIGS. 18-24 illustrate various examples of an electrode including the non-stick surface.

FIG. 18 illustrates an example where the non-stick surface 1825 is applied along the perimeter of the electrode 1824. The non-stick surface 1825 can be applied along the entire non-stick surface 1825 or along a portion of the perimeter.

FIG. 19 illustrates an example where the non-stick surface 1925 is applied along a portion of perimeter of the electrode 1924 and along a midsection of the electrode 1924. For example, various portions of the electrode 1924 surface that are prone to build-up can be subject to the non-stick surface 1925.

FIG. 20 illustrates an example where the non-stick surface 2025 is applied along a portion of perimeter of the electrode 2024. However, as seen in this example, the portions of the electrode surface are non-consistent along the perimeter. FIG. 21 illustrates an example where the non-stick surface 2125 is applied to a portion of the overall surface of the electrode 2124. While shown as discrete circular segments, the non-stick surface 2125 can have any shape and can include a plurality of similar shapes or non-similar shapes. Further, the overall percentage of surface area that the non-stick surface 2124 occupies can vary. For example, at least about 10 percent (%) or more of the total surface area of the electrode 2124 can include the non-stick surface 2125. As shown in FIG. 22, the non-stick surface 2225 covers the entire surface of the electrode 2224.

FIGS. 23 and 24 illustrate the non-stick coating 127 that can be applied to the jaw body 122 of the forceps. As seen in FIG. 23, the non-stick coating 127 is applied to an entire external surface of the jaw body 122. FIG. 24 illustrates and example where the non-stick coating 127 is applied to a portion of the external surface of the jaw body 122.

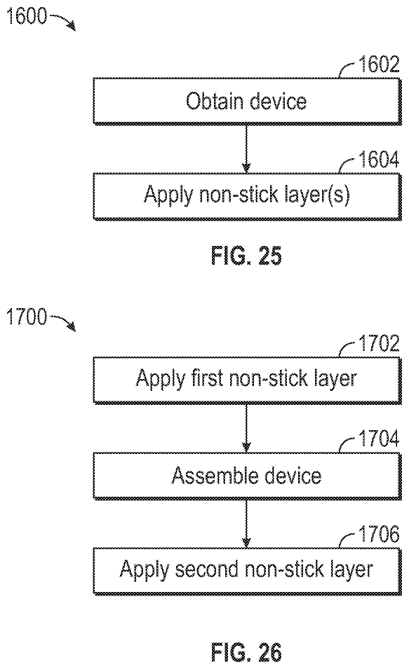

FIG. 25 is a flow chart depicting a method 1600 of applying non-stick coating(s) a surgical device. The method 1600 can include obtaining an electrosurgery cutting device (block 1602) and applying a non-stick layer(s).

The method 1600 can include etching the surface of electrodes of a bipolar cutting device with a non-stick layer 125, such that the layer at least partially covers the electrode. Application of the coating or layer can be done, for example, by chemical etching or laser etching as discussed herein. Additionally, non-stick coating 127 can be applied to the external surface of the jaw bodies. For example, the non-stick coating 127 can be applied by, e.g., spraying or depositing the non-stick coating 127. The electrode surface can be masked off to prevent the non-stick coating 127 from interfering with the electrode surface and/or non-stick layer 125.

In some cases, the coating can be produced in a uniform thickness of about 1 nm to about 300 nm, of about 5 nm to about 200 nm, or of about 10 nm to about 100 nm. In some cases, the coating can be produced in a pattern, such as to create hydrophobic pillars on the jaw body. In some cases, the coating can fully or partially cover the jaw body.

The method 1700 of FIG. 26 can include etching the surface of electrodes of a bipolar cutting device with a non-stick layer 125 (1702) such that the layer at least partially covers the electrode prior to the electrode being assembled with the forceps. After the application of the non-stick layer 125, the electrode can be assembled into the forceps (1704). The non-stick coating 127 can then be applied (1704), as discussed herein.

Several modification/application techniques may be used to form the coating, optionally including hydrophobic pillars. In one example, a sol-gel process can be used. Advantages of sol-gel application include the ability to coat more complex surfaces with high quality films. Challenges of sol-gel may include brittleness, limited thickness options, and induced mechanical stresses in the coating.

In one example, a cold spray process can be used. Advantages of cold spray application include the ability to coat at lower temperatures, with low deterioration, low oxidation, and low defects. Challenges of cold spray may include high energy needed for application, high cost, and a limited number of compatible substrates.

In one example, a chemical vapor deposition (CVD) process can be used. Advantages of CVD application include a high quality coating, high control of thickness, and the ability to coat complex surfaces. Challenges of CVD may include high temperature requirements, and high cost.

In one example, a physical vapor deposition (PVD) process can be used. Advantages of PVD application include the ability to coat inorganic compounds, ecological friendly processes, and a wide variety of available coating materials. Challenges of PVD may include high vacuum chamber requirements and high cost.

In one example, a thermal spray process can be used. Advantages of thermal spray application include a large selection of compatible coating materials and substrate materials, and low cost. Challenges of thermal spray may include difficulty in forming thick coatings, low adhesion issues of coatings, and ecologically unfriendly process steps.

In one example, an in-situ polymerization process can be used. Advantages of in-situ polymerization include the ability to coat with insoluble polymers. Challenges of in-situ polymerization may include process complexity, high cost, and limited potential for large scale production.

In one example, a spin coating process can be used. Advantages of spin coating include high quality coatings, fast drying times, and controllable thicknesses. Challenges of spin coating may include difficulty coating small surfaces and requirements of a smooth surface.

In one example, a dip coating process can be used. Advantages of dip coating include the ability to coat complex surfaces and the ability for large scale production. Challenges of dip coating may include undesirable solvent requirements, and limitations of only soluble polymer coatings.

In one example, an electrodeposition process can be used. Advantages of electrodeposition include high quality coatings at low cost. Challenges of electrodeposition may include long process times, and conductive substrate requirements.

Medical devices having a non-stick coating including a laser etched surface along a portion of the electrode along with the coatings, described herein, along the external surfaces of the jaw body, provide a robust and efficient non-stick medical device that show reduced adhesion over other non-textured coatings for bio materials including, but not limited to, tissues, blood, fats, and/or other biological materials. Application of the non-stick coatings and surfaces to other surfaces of medical devices apart from the components discussed herein may further provide advantages such as reduced friction and reduced adhesion where desired.

The benefits of the systems and methods of the present disclosure can include tissue adherence resistance with non-stick coatings formed from the liquidphobic structures disclosed herein.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more,” in this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

### Exemplary Aspects.

The following exemplary aspects are provided, the numbering of which is not to be construed as designating levels of importance:

Aspect 1 provides an electrosurgical device, comprising:
at least one jaw member having an electrically conductive tissue sealing plate configured to operably couple to a source of electrosurgical energy for treating tissue; and
a non-stick coating formed from a liquidphobic structure disposed on at least a portion of the electrically conductive tissue sealing plate.

Aspect 2 provides the electrosurgical device of Aspect 1, wherein the liquidphobic structure includes a textured surface including a micro-topography.

Aspect 3 provides the electrosurgical device of any one of Aspects 1 or 2, wherein the micro-topography includes micro-pillars.

Aspect 4 provides the electrosurgical device of Aspect 3, wherein the micro-pillars have a height within a range of about 10 microns to about 250 microns.

Aspect 5 provides the electrosurgical device of any one of Aspects 3 or 4, wherein the micro-pillars have a width within a range of about 70 microns to about 400 microns.

Aspect 6 provides the electrosurgical device of any one of Aspects 1-5, wherein the micro-pillars have a center-to-center distance within a range of about 120 microns to about 500 microns.

Aspect 7 provides the electrosurgical device of any one of Aspects 2-6, wherein the liquidphobic structure further includes:
a nano-topography superimposed onto the micro-topography.

Aspect 8 provides the electrosurgical device of Aspect 7, wherein the nano-topography includes nano-pillars formed onto the micro-topography.

Aspect 9 provides the electrosurgical device of Aspect 8, wherein nano-pillars have a height within a range of about 2 nanometers to about 20 nanometers.

Aspect 10 provides the electrosurgical device any one of Aspects 8 or 9, wherein the nano-pillars have a width within a range of about 50 nm to about 300 nm.

Aspect 11 provides the electrosurgical device of any one of Aspects 8-10, wherein the nano-pillars have a center-to-center distance within a range of about 120 nm to about 350 nm.

Aspect 12 provides the electrosurgical device any one of Aspects 7-11, wherein the nano-topography includes tube-like structures formed into the micro-topography.

Aspect 13 provides the electrosurgical device any one of Aspects 7-12, wherein dimensions of the nano-pillars are nonuniform and dimensions of the micro-pillars are nonuniform.

Aspect 14 provides the electrosurgical device any one of Aspects 2-13, wherein the liquidphobic structure further includes a plurality of nanotubes deposited on the micro-topography.

Aspect 15 provides an electrosurgical device, comprising:
a pair of opposing jaw members, each of the opposing jaw members including:
   an electrically conductive tissue sealing plate configured to operably couple to a source of electrosurgical energy for treating tissue;
   a support base configured to support the tissue sealing plate; and
   an insulative housing configured to secure the tissue sealing plate to the support base; and
a non-stick coating disposed on at least a portion of at least one of the opposing jaw members, the non-stick coating formed from a liquidphobic structure.

Aspect 16 provides the electrosurgical device of Aspect 15, wherein the liquidphobic structure includes a textured surface including a micro-topography.

Aspect 17 provides the electrosurgical device of Aspect 16, wherein the micro-topography includes micro-pillars.

Aspect 18 provides the electrosurgical device of Aspect 17, wherein the micro-pillars have a height within a range of about 10 microns to about 2250 microns.

Aspect 19 provides the electrosurgical device any one of Aspects 17 or 18, wherein the micro-pillars have a width within a range of about 70 microns to about 400 microns

Aspect 20 provides the electrosurgical device any one of Aspects 17-19, wherein the micro-pillars have a center-to-center distance within a range of about 120 microns to about 500 microns.

Aspect 21 provides the electrosurgical device any one of Aspects 16-20, wherein the liquidphobic structure further includes:
a nano-topography superimposed onto the micro-topography.

Aspect 22 provides the electrosurgical device of Aspect 21, wherein the nano-topography includes nano-pillars formed onto the micro-topography.

Aspect 23 provides the electrosurgical device of Aspect 22, wherein nano-pillars have a height within a range of about 2 nanometers to about 20 nanometers.

Aspect 24 provides the electrosurgical device any one of Aspects 22 or 23, wherein the nano-pillars have a width within a range of about 50 nm to about 300 nm.

Aspect 25 provides the electrosurgical device any one of Aspects 22-24, wherein the nano-pillars have a center-to-center distance within a range of about 120 nm to about 350 nm.

Aspect 26 provides the electrosurgical device any one of Aspects 21-25, wherein the nano-topography includes tube-like structures formed into the micro-topography.

Aspect 27 provides the electrosurgical device any one of Aspects 21-26, wherein dimensions of the nano-pillars are nonuniform and dimensions of the micro-pillars are nonuniform.

Aspect 28 provides the electrosurgical device any one of Aspects 16-27, wherein the liquidphobic structure further includes a plurality of nanotubes deposited on the micro-topography.

Aspect 29 provides an electrosurgical device, comprising:
a pair of opposing jaw members, each of the opposing jaw members including:
   an electrically conductive tissue sealing plate configured to operably couple to a source of electrosurgical energy for treating tissue;
   a support base configured to support the tissue sealing plate; and
   an insulative housing configured to secure the tissue sealing plate to the support base;
a first coating disposed on at least a portion of the electrically conductive tissue sealing plate of at least one of the opposing jaw members, the first coating configured to increase the durability of the at least one of the opposing jaw members; and
a liquidphobic structure disposed on at least a portion of at least one of the opposing jaw members including a portion of the first coating.

Aspect 30 provides the electrosurgical device of Aspect 29, wherein the first coating is selected from chromium nitride and titanium nitride.

Aspect 31 provides the electrosurgical device of Aspect 30, wherein the liquidphobic structure is a textured portion of the first coating.

Aspect 32 provides the electrosurgical device of Aspect 31, wherein the textured portion includes a micro-topography.

Aspect 33 provides the electrosurgical device of Aspect 32, wherein the micro-topography includes micro-pillars.

Aspect 34 provides the electrosurgical device of Aspect 33, wherein the micro-pillars have a height within a range of about 10 microns to about 200 microns.

Aspect 35 provides the electrosurgical device any one of Aspects 33 or 34, wherein the micro-pillars have a width within a range of about 70 microns to about 400 microns.

Aspect 36 provides the electrosurgical device any one of Aspects 33-35, wherein the micro-pillars have a center-to-center distance within a range of about 120 microns to about 500 microns.

Aspect 37 provides the electrosurgical device any one of Aspects 16-36, wherein the liquidphobic structure further includes:
a nano-topography superimposed onto the micro-topography.

Aspect 38 provides the electrosurgical device of Aspect 37, wherein nano-topography includes nano-pillars.

Aspect 39 provides the electrosurgical device of Aspect 38, wherein nano-pillars have a height within a range of about 2 nanometers to about 20 nanometers.

Aspect 40 provides the electrosurgical device of Aspect 38, wherein the nano-pillars have a width within a range of about 50 nm to about 300 nm.

Aspect 41 provides the electrosurgical device any one of Aspects 38 or 39, wherein the nano-pillars have a center-to-center distance within a range of about 120 nm to about 350 nm.

Aspect 42 provides the electrosurgical device any one of Aspects 37-41, wherein the nano-topography includes tube-like structures formed into the micro-topography.

Aspect 43 provides the electrosurgical device any one of Aspects 37-42, wherein dimensions of the micro-topography are nonuniform and dimensions of the nano-topography are nonuniform.

Aspect 44 provides the electrosurgical device any one of Aspects 32-43, wherein the liquidphobic structure further includes a plurality of nanotubes deposited on the micro-topography.

Aspect 45 provides a method of manufacturing an electrosurgical device, the method comprising:
coupling an electrically conductive sealing plate to a support base to form a jaw member; and
forming a liquidphobic structure on at least a portion of the electrically conductive sealing plate, wherein the liquidphobic structure reduces sticking of the tissue to the electrically conductive sealing plate as compared to an electrically conductive sealing plate without the liquidphobic structure during delivery of electrosurgical energy.

Aspect 46 provides the method of Aspect 45, wherein forming the liquidphobic structure includes texturing a portion of at least the portion of the electrically conductive sealing plate.

Aspect 47 provides the method of Aspect 46, wherein texturing the portion of at least the portion of the electrically conducive sealing plate including forming micro-pillars.

Aspect 48 provides the method of Aspect 47, wherein the micro-pillars are formed using at least one technique selected from laser etching, chemical etching, and micro-machining.

Aspect 49 provides the method any one of Aspects 47 or 48, wherein texturing the portion of the at least the portion of the electrically conductive sealing plate includes forming a nano-topography on the micro-topography.

Aspect 50 provides the method any one of Aspects 48 or 49, wherein the nano-topography is formed by depositing a layer of a plurality of nanotubes.

Aspect 51 provides the method any one of Aspects 48-50, wherein the nano-topography is formed by forming a plurality of tube-like structures on the microtopography.

Aspect 52 provides the method of Aspect 51, wherein the tube-like structures are formed by potentiostatic anodization.

Aspect 53 provides the method any one of Aspects 45-521, further comprising:
overmolding an insulative material about the support base to secure the electrically conductive sealing plate thereto.

Aspect 54 provides the method of Aspect 53, wherein the insulative material includes the liquidphobic structure.

Aspect 55 provides the method of Aspect 54, wherein the liquidphobic structure includes a micro-topography.

Aspect 56 provides the method of Aspect 55, wherein the micro-topography is formed during the overmolding.

Aspect 57 provides the method of any one of Aspects 45-56, further including coupling an electrical lead to the electrically conductive sealing surface, the electrical lead configured to connect the electrically conductive sealing surface to an energy source.

Aspect 58 provides a method of manufacturing an electrosurgical device, the method comprising:
applying a first coating to at least a portion of an electrically conductive sealing plate to form a coated electrically conductive sealing plate;
coupling the coated electrically conductive sealing plate to a support base to form a jaw member; and
forming a liquidphobic structure over at least a portion of the coated electrically conductive sealing plate, wherein the liquidphobic structure reduces sticking of the tissue to the electrically conductive sealing plate as compared to an electrically conductive sealing plate without the liquidphobic structure during delivery of electrosurgical energy.

Aspect 59 provides the method of Aspect 58, wherein forming the liquidphobic structure includes texturing a portion of at least the portion of the first coating.

Aspect 60 provides the method of Aspect 59, wherein texturing the portion of at least the portion of the first coating includes forming micro-pillars.

Aspect 61 provides the method of Aspect 60, wherein the micro-pillars are formed using at least one technique selected from laser etching, chemical etching, and micro-machining.

Aspect 62 provides the method any one of Aspects 59-62, wherein texturing the portion of the at least the portion of the first coating includes forming a nano-topography on the micro-topography.

Aspect 63 provides the method of Aspect 62, wherein the nano-topography is formed by depositing a layer of a plurality of nanotubes.

Aspect 64 provides the method any one of Aspects 62 or 63, wherein the nano-topography is formed by forming a plurality of tube-like structures on the microtopography.

Aspect 65 provides the method of Aspect 47, further comprising:
overmolding an insulative material about the support base to secure the electrically conductive sealing plate thereto.

Aspect 66 provides the method of Aspect 65, wherein the insulative material includes the liquidphobic structure.

Aspect 67 provides the method of Aspect 66, wherein the liquidphobic structure includes a micro-topography.

Aspect 68 provides the method of Aspect 67, wherein the micro-topography is formed during the overmolding.

Aspect 69 provides the method of any one of Aspects 65-68, further including coupling an electrical lead to the electrically conductive sealing surface, the electrical lead configured to connect the electrically conductive sealing surface to an energy source.

Aspect 70 provides a device, comprising:
at least one jaw member having a surface configured to contact tissue; and
a non-stick coating formed from a liquidphobic structure disposed on at least a portion of the surface.

Aspect 71 provides an electrosurgical device, comprising:
at least one jaw member having a jaw body and an electrically conductive tissue sealing plate configured to operably couple to a source of electrosurgical energy for treating tissue;
a first non-stick coating disposed on at least a portion of the electrically conductive tissue sealing plate; and
a second nonstick coating disposed on at least a portion of an external surface of the jaw body, the second non-stick coating different from the first non-stick coating.

Aspect 72 provides the electrosurgical device of Aspect 71, wherein the first non-stick coating has a surface adherence to tissue that is less than a surface adherence of the electrically conductive tissue sealing plate that does not include the non-stick coating.

Aspect 73 provides the electrosurgical device according to any one of Aspects 71 and 72, wherein the second non-stick coating comprises a material having a surface adherence to tissue that is less than a surface adherence to tissue of the material of the jaw body.

Aspect 74 provides the electrosurgical device according to any one of Aspects 71-73, wherein the first non-stick coating is an etched surface including micropillars.

Aspect 75 provides the electrosurgical device of Aspect 74, wherein the first non-stick coating is a hydrophobic surface structure.

Aspect 76 provides the electrosurgical device according to any one of Aspects 71-75, wherein the first non-stick coating is formed along a portion of the perimeter of a surface of the electrically conductive tissue sealing plate.

Aspect 77 provides the electrosurgical device according to any one of Aspects 71-76, wherein the first non-stick coating is at least 10 percent of a total surface area of the electrically conductive tissue sealing plate.

Aspect 78 provides the electrosurgical device according to any one of Aspects 71-77, wherein the first non-stick coating is at least 50 percent of a total surface area of the electrically conductive tissue sealing plate.

Aspect 79 provides the electrosurgical device according to any one of Aspects 71-78, wherein the second non-stick coating is selected from one of polydimethylsiloxane, hexadimethylsiloxane, and tetramethyldisiloxane.

Aspect 80 provides the electrosurgical device according to Aspect 79, wherein the second non-stick coating has a thickness within a range of about 10 nm to about 300 nm.

Aspect 81 provides the electrosurgical device according to Aspect 80, where the non-stick coating has a thickness within a range of about 200 nm to about 250 nm.

Aspect 82 provides the electrosurgical device according to any one of Aspects 71 through 79, wherein the second non-stick coating has a substantially uniform thickness.

Aspect 83 provides the electrosurgical device according to any one of Aspects 71 through 79, wherein the second non-stick coating has a non-uniform thickness.

Aspect 84 provides the electrosurgical device according to any one of Aspects 71 through 83, wherein the electrically conductive tissue sealing plate is formed of stainless steel.

Aspect 85 provides an electrosurgical device, comprising:
at least one jaw member having a jaw body and an electrically conductive tissue sealing plate configured to operably couple to a source of electrosurgical energy for treating tissue;
a first non-stick surface formed on at least a portion of the electrically conductive tissue sealing plate, the first non-stick surface including etched micropillars; and
a second non-stick coating disposed on at least a portion of an external surface of the jaw body, the second non-stick coating selected from one of polydimethylsiloxane, hexadimethylsiloxane, and tetramethyldisiloxane.

Aspect 86 provides a method comprising:
applying a first non-stick layer to a tissue sealing plate of an end effector in a surgical device, wherein the non-stick layer comprises a hydrophobic surface structure; and
applying a second non-stick layer to a portion of an external surface of a jaw body of an end effector in a surgical device, wherein the second non-stick layer comprises a deposited coating.

Aspect 87 provides the method according to Aspect 86, wherein applying the first non-stick layer comprises etching the hydrophobic surface structure.

Aspect 88 provides the method of Aspect 87, wherein the hydrophobic surface structures includes micropillars.

Aspect 89 provides the method according to any one of Aspects 86 through 88, wherein depositing the second non-stick layer comprises applying a coating to the external surface of the jaw body.

Aspect 90 provides the method according to any one of Aspects 16 through 18, the second non-stick layer has a thickness within a range of about 10 nm to about 300 nm.

Aspect 91 provides a surgical probe comprising:
shaft having a distal portion and a proximal portion;
a probe tip extending distally from the distal portion of the shaft, the probe tip comprising at least one electrically conductive electrode configured to coagulate tissue; and
a coating at least partially covering the at least one electrode, wherein the coating comprises a coating material having a surface adherence to tissue that is less than a surface adherence to tissue of the material of the at least one electrode.

Aspect 92 provides the probe of Aspect 91, wherein the at least one electrically conductive electrode comprises a first electrode and a second electrode having a polarity opposite the first electrode, wherein the first and second electrodes alternatively spiral distally along the probe tip.

Aspect 93 provides the probe of any one of Aspects 91-93, wherein the at least one electrode comprises an electrode formed as a surgical needle.

Aspect 94 provides the probe of any one of Aspect 91-93, wherein the coating has a thickness of about 1 nm to about 300 nm.

Aspect 95 provides the probe of Aspect 94, wherein the coating has a thickness of about 5 nm to about 250 nm.

Aspect 96 provides the probe of Aspect 95, wherein the coating has a thickness of about 10 nm to about 100 nm.

Aspect 97 provides the probe of any one of Aspects 91-96, wherein the coating comprises a uniform thickness.

Aspect 98 provides the probe of any one of Aspects 91-97, wherein the coating comprises an impedance of less than about 10 Ohms.

Aspect 99 provides the probe of any one of Aspects 91-98, wherein the coating comprises one or more polysiloxanes, fluorosilanes, or combinations thereof.

Aspect 100 provides the probe of any one of Aspects 91-99, wherein the coating comprises a superhydrophobic material.

Aspect 101 provides the probe of Aspect 100, wherein the coating comprises one or more pillars superimposed on the at least one electrode.

Aspect 102 provides the probe of any one of Aspects 100 or 101, wherein the coating has a surface energy less than that of the at least one conductive electrode.

Aspect 103 provides the probe of any one of Aspects 100-102, wherein the coating has a lower surface energy than that of the at least one conductive electrode.

Aspect 104 provides the probe of any one of Aspects 100-103, wherein the coating comprises an electrically insulating material or a non-conductive material.

Aspect 105 provides the probe of any one of Aspects 100-104, wherein the coating comprises an electrical conductivity of less than that of the at least one electrically conductive electrode.

Aspect 106 provides the probe of any one of Aspects 100-105, wherein the coating comprises a coefficient of friction of about 0.05 to about 0.15.

Aspect 107 provides the probe of any one of Aspects 100-106, wherein the coating overlays a portion of the at least one electrode.

Aspect 108 provides the probe of any one of Aspects 100-107, wherein the coating is a polymeric coating.

Aspect 109 provides a method comprising:
coating a bipolar electrode on a surgical probe with a non-stick coating, such that the coating at least partially covers the bipolar electrode.

Aspect 110 provides the method of Aspect 109, wherein coating comprises chemical etching, laser etching, chemical bombardment, or printing.

1. An electrosurgical device, comprising:
at least one jaw member having an electrically conductive tissue sealing plan; configured to operably couple to a source of electrosurgical energy for treating tissue; and
a non-stick coating formed from a liquidphobic structure, comprising a textured surface including a micro-topography, disposed on at least a portion of the electrically conductive tissue sealing plate.

2. The electrosurgical device of claim 1, wherein the micro-topography includes micro-pillars.

3. The electrosurgical device of claim 2, wherein the micro-pillars have a height within a range of about 10 microns to about 250 microns.

4. The electrosurgical device of claim 3, wherein the micro-pillars have a width within a range of about 70 microns to about 400 microns.

5. The electrosurgical device of claim 1, wherein the micro-pillars have a center-to-center distance within a range of about 120 microns to about 500 microns.

6. The electrosurgical device of claim 1, wherein the liquidphobic structure further includes:
a nano-topography superimposed onto the micro-topography.

7. The electrosurgical device of claim 6, wherein the nano-topography includes nano-pillars formed onto the micro-topography.

8. The electrosurgical device of claim 7, wherein nano-pillars have a height within a range of about 2 nanometers to about 20 nanometers.

9. The electrosurgical device of claim 7, wherein the nano-pillars have a width within a range of about 50 nm to about 300 nm.

10.-12. (canceled)

13. An electrosurgical device, comprising:
at least one jaw member having a jaw body and an electrically conductive tissue sealing plate configured to operably couple to a source of electrosurgical energy for treating tissue;
a first non-stick coating disposed on at least a portion of the electrically conductive tissue sealing plate; and
a second non-stick coating disposed on at least a portion of an external surface of the jaw body, the second non-stick coating different from the first non-stick coating.

14. The electrosurgical device of claim 13, wherein the first non-stick coating has a surface adherence to tissue that is less than a surface adherence of the electrically conductive tissue sealing plate that does not include the non-stick coating.

15. The electrosurgical device of claim 13, wherein the second non-stick coating comprises a material having a surface adherence to tissue that is less than a surface adherence to tissue of the material of the jaw body.

16.-19. (canceled)

20. The electrosurgical device of claim 13, wherein the first non-stick coating is at least 50 percent of a total surface area of the electrically conductive tissue sealing plate.

21. The electrosurgical device of claim 13, wherein the second non-stick coating is selected from one of polydimethylsiloxane, hexadimethylsiloxane, and tetramethyldisiloxane.

22.-26. (canceled)

27. A surgical probe comprising:
a shaft having a distal portion and a proximal portion;
a probe tip extending distally from the distal portion of the shaft, the probe tip comprising at least one electrically conductive electrode configured to coagulate tissue; and
a coating at least partially covering the at least one electrode, wherein the coating comprises a coating material having a surface adherence to tissue that is less than a surface adherence to tissue of the material of the at least one electrode.

28. The probe of claim 27, wherein the at least one electrically conductive electrode comprises a first electrode and a second electrode having a polarity opposite the first electrode, wherein the first and second electrodes alternatively spiral distally along the probe tip.

29. The probe of claim 27, wherein the at least one electrode comprises an electrode formed as a surgical needle.

30.-33. (canceled)

34. The probe of claim 27, wherein the coating comprises an impedance of less than about 10 Ohms.

35. The probe of claim 27, wherein the coating comprises one or more polysiloxanes, fluorosilanes, or combinations thereof.

36. The probe of claim 27, wherein the coating comprises a superhydrophobic material.

37.-43. (canceled)
